(19) United States
(12) Patent Application Publication
(10) Pub. No.: US 2025/0331803 A1
(43) Pub. Date: Oct. 30, 2025

(54) AUTOMATIC COLLIMATOR INSTALLATION SYSTEMS AND METHODS

(71) Applicant: SHANGHAI UNITED IMAGING HEALTHCARE CO., LTD., Shanghai (CN)

(72) Inventors: Beien WANG, Shanghai (CN); Li FANG, Shanghai (CN)

(73) Assignee: SHANGHAI UNITED IMAGING HEALTHCARE CO., LTD., Shanghai (CN)

(21) Appl. No.: 19/256,039

(22) Filed: Jun. 30, 2025

Related U.S. Application Data
(63) Continuation-in-part of application No. 18/052,945, filed on Nov. 7, 2022, now Pat. No. 12,343,882.

(30) Foreign Application Priority Data
Nov. 8, 2021 (CN) 202111315533.5
Dec. 31, 2024 (CN) 202411998538.6

(51) Int. Cl. A61B 6/58; A61B 6/00; A61B 6/06; A61B 50/13

(52) U.S. Cl. CPC A61B 6/586; A61B 6/06; A61B 6/4494; A61B 6/547; A61B 6/583; A61B 50/13

(57) ABSTRACT

The present disclosure is related to automatic installation systems and methods. An automatic collimator installation method may include obtaining an installation instruction for installing a target collimator into a medical scanner, identifying, from a plurality of collimators stored in a tool storage device, the target collimator based on the installation instruction; using a cart to transport the target collimator from the tool storage device to the medical scanner; and automatically installing, using the cart, the target collimator into the medical scanner.

### CROSS-REFERENCE TO RELATED APPLICATIONS

This application claims priority of Chinese Patent Application No. 202411998538.6, filed on Dec. 31, 2024, and is a continuation in part of U.S. application Ser. No. 18/052,945, filed on Nov. 7, 2022, which claims priority of Chinese Patent Application No. 202111315533.5, filed on Nov. 8, 2021, the contents of each of which are hereby incorporated by reference.

### TECHNICAL FIELD

This disclosure generally relates to collimator technology, and more particularly, relates to automatic installation systems and methods.

### BACKGROUND

Nuclear medicine functional imaging techniques (e.g., single-photon emission computed tomography (SPECT)) are widely used in medical diagnosis. A SPECT device generally has a plurality of different collimators for positioning different radioactive tracers. However, the plurality of collimators are usually manually installed or uninstalled, which wastes a lot of time and manpower. Thus, it is desirable to develop automatic installation systems and methods.

### SUMMARY

According to an aspect of the present disclosure, an automatic installation system is provided. The system may include a cart, a storage medium including a set of instructions, and at least one processor configured to communicate with the storage medium. When executing the set of instructions, the at least one processor is configured to direct the system to perform operations including:

In some embodiments, the using the cart to transport the target collimator from the tool storage device to the medical scanner may include: using the cart to transport, based on a predetermined route, the target collimator from the tool storage device to a predetermined location of the medical scanner; and using the cart to align, based on a navigation algorithm, the target collimator with a detector of the medical scanner.

In some embodiments, the navigation algorithm may include at least one of: a laser navigation algorithm, a visual navigation algorithm, or an inertial navigation algorithm.

In some embodiments, the automatically installing, by the cart, the target collimator into the medical scanner may include: automatically installing, by a locating module of the cart, the target collimator onto a detector of the medical scanner.

In some embodiments, the locating module includes a locating hole or a locating pin.

In some embodiments, the identifying, from the plurality of collimators stored in the tool storage device, the target collimator based on the installation instruction may include: obtaining a target identifier of the target collimator based on the installation instruction; identifying a plurality of identifiers respectively corresponding to the plurality of collimators; and identifying the target collimator by matching the target identifier with one of the plurality of identifiers respectively corresponding to the plurality of collimators.

In some embodiments, the plurality of identifiers of the plurality of collimators may be identified according to at least one of: a mechanical switch, a proximity switch, a photoelectric switch, an electromagnetic sensor, a radiofrequency identification tag, or a unique symbol.

In some embodiments, the operations may further include: automatically uninstalling, using the cart, an installed collimator from the medical scanner based on the installation instruction; and using the cart to transport the installed collimator from the medical scanner to the tool storage device.

In some embodiments, the target collimator may include at least one of: a pinhole collimator, a parallel hole collimator, a fan-beam collimator, a cone-beam collimator, or a slit-slat collimator.

In some embodiments, the automatic installation system may further comprise the tool storage device configured to store the plurality of collimators, and the tool storage device is away from the medical scanner.

In some embodiments, the operations may further include: obtaining a scan protocol; and determining the installation instruction based on the scan protocol.

According to another aspect of the present disclosure, a cart may include a mechanical arm, a cart body, a processor, and a storage medium including a set of instructions. The processor may be configured to communicate with the storage medium. when executing the set of instructions, the processor may be configured to direct the cart to perform operations including: obtaining an installation instruction for installing a target collimator into a medical scanner; identifying, from a plurality of collimators stored in a tool storage device, the target collimator based on the installation instruction; griping, using the mechanical arm, the target collimator from the tool storage device to the cart body; controlling the cart body to transport the target collimator from the tool storage device to the medical scanner; and automatically installing, using the mechanical arm, the target collimator into the medical scanner.

According to another aspect of the present disclosure, an automatic collimator installation method may include obtaining an installation instruction for installing a target collimator into a medical scanner; identifying, from a plurality of collimators stored in a tool storage device, the target collimator based on the installation instruction; using a cart to transport the target collimator from the tool storage device to the medical scanner; and automatically installing, using the cart, the target collimator into the medical scanner.

According to still another aspect of the present disclosure, a non-transitory computer readable medium may include at least one set of instructions. When executed by at least one processor of a computing device, the at least one set of instructions may cause the at least one processor to effectuate a method. The method may include obtaining an installation instruction for installing a target collimator into a medical scanner; identifying, from a plurality of collimators stored in a tool storage device, the target collimator based on the installation instruction; using a cart to transport the target collimator from the tool storage device to the medical scanner; and automatically installing, using the cart, the target collimator into the medical scanner.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary automatic installation system according to some embodiments of the present disclosure;

FIG. 2 is a schematic diagram illustrating exemplary hardware and/or software components of an exemplary computing device on which a processing device may be implemented according to some embodiments of the present disclosure;

FIG. 3 is a schematic diagram illustrating exemplary hardware and/or software components of an exemplary mobile device according to some embodiments of the present disclosure;

FIG. 4 is a block diagram illustrating an exemplary processing device according to some embodiments of the present disclosure;

FIG. 5 is a flowchart illustrating an exemplary process for automatically installing a target collimator according to some embodiments of the present disclosure;

FIG. 6 is a schematic diagram illustrating an exemplary automatic installation system according to some embodiments of the present disclosure;

FIG. 7 is a schematic diagram illustrating an exemplary automatic installation system according to some embodiments of the present disclosure;

FIG. 8 is a schematic diagram illustrating an exemplary automatic installation system according to some embodiments of the present disclosure;

FIG. 9 is a flowchart illustrating an exemplary process for transporting a target collimator according to some embodiments of the present disclosure;

FIG. 10 is a flowchart illustrating an exemplary process for identifying a target collimator according to some embodiments of the present disclosure;

FIG. 11 is a flowchart illustrating an exemplary process for uninstalling an installed collimator according to some embodiments of the present disclosure;

FIG. 12 is a flowchart illustrating an exemplary process for determining an installation instruction according to some embodiments of the present disclosure;

FIG. 13 is a flowchart illustrating an exemplary process for automatically quality control of a medical device according to some embodiments of the present disclosure;

FIG. 14 is a flowchart illustrating an exemplary process for automatically adjusting a position of a QC tool according to some embodiments of the present disclosure;

FIG. 15 is a diagram illustrating an exemplary process for automatically QC according to some embodiments of the present disclosure; and

FIG. 16 is a diagram illustrating another exemplary process for automatically QC according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Also, the term “exemplary” is intended to refer to an example or illustration.

It will be understood that the terms “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

Spatial and functional relationships between elements are described using various terms, including “connected,” “attached,” and “mounted.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, attached, or positioned to another element, there are no intervening elements present. Other words used to describe the relationship between elements should be interpreted in a like fashion (e.g., “between,” versus “directly between,” “adjacent,” versus “directly adjacent,” etc.).

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “anatomical structure” in the present disclosure may refer to gas (e.g., air), liquid (e.g., water), solid (e.g., stone) cell, tissue, organ of a subject, or any combination thereof, which may be displayed in an image and really exist in or on the subject's body. The term “region,” “location,” and “area” in the present disclosure may refer to a location of an anatomical structure shown in the image or an actual location of the anatomical structure existing in or on the subject's body, since the image may indicate the actual location of a certain anatomical structure existing in or on the subject's body. The term “an image of a subject” may be referred to as the subject for brevity.

For illustration purposes, the following description is provided to help better understanding an image registration process. It is understood that this is not intended to limit the scope of the present disclosure. For persons having ordinary skills in the art, a certain amount of variations, changes and/or modifications may be deducted under the guidance of the present disclosure. Those variations, changes and/or modifications do not depart from the scope of the present disclosure.

An aspect of the present disclosure relates to automatic installation systems and methods. As used herein, the word “automatically” or “automatic” may refer that a process is performed in a mechanical manner without any human assistance. In some embodiments, the systems and methods may identify, from a plurality of collimators stored in a tool storage device, a target collimator based on an installation instruction. The systems and methods may use a cart to transport the target collimator from the tool storage device to a medical scanner (e.g., a SPECT device, a CT-SPCT device, etc.), and automatically install, using the cart, the target collimator into the medical scanner. In some embodiments, the cart may automatically identify the target collimator by matching a target identifier of the target collimator with one of the plurality of identifiers respectively corresponding to the plurality of collimators. The cart may transport the target collimator from the tool storage device to a predetermined location of the medical scanner based on a predetermined route. The cart may align, based on a navigation algorithm (e.g., a laser navigation algorithm, a visual navigation algorithm, or an inertial navigation algorithm, etc.), the target collimator with a detector of the medical scanner. The cart may automatically install, by a locating module (e.g., a locating hole or a locating pin) of the cart, the target collimator onto a detector of the medical scanner. According to some embodiments of the present disclosure, the target collimator may be automatically identified, installed, or uninstalled without any human assistance. The accuracy of identifying the target collimator and the installation efficiency of collimators may be improved.

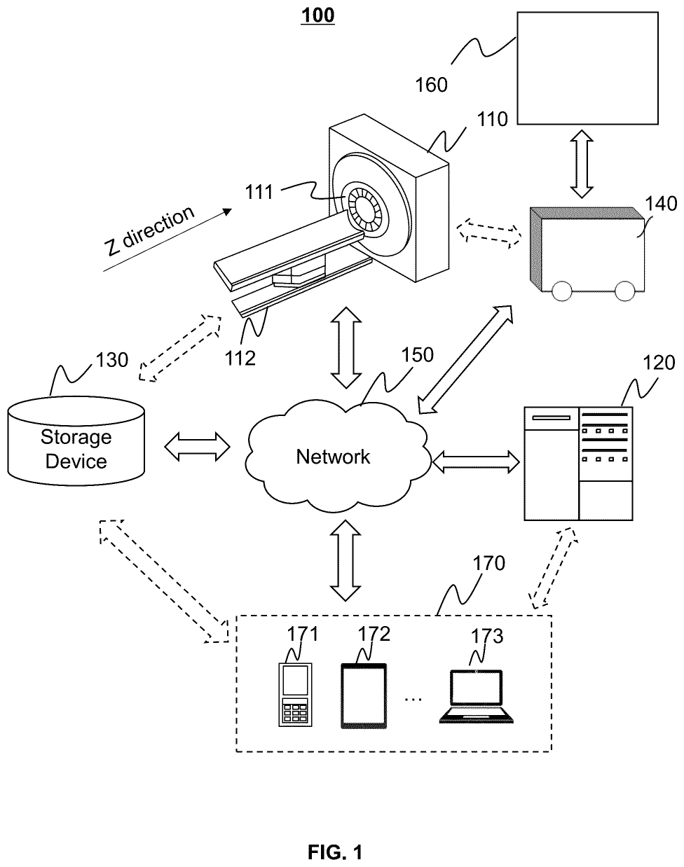

FIG. 1 is a schematic diagram illustrating an exemplary automatic installation system 100 according to some embodiments of the present disclosure. As shown, the automatic installation system 100 may include a medical device 110, a processing device 120, a storage device 130, a cart 140, a network 150, a tool storage device 160, and one or more terminal(s) 170. In some embodiments, the medical device 110, the processing device 120, the storage device 130, the cart 140, the tool storage device 160, and/or the terminal(s) 170 may be connected to and/or communicate with each other via a wireless connection (e.g., the network 150), a wired connection, or a combination thereof. The automatic installation system 100 may include various types of connections between its components. For example, the medical device 110 may be connected to the processing device 120 through the network 150, or connected to the processing device 120 directly as illustrated by the bidirectional dotted arrow connecting the medical device 110 and the processing device 120 in FIG. 1. As another example, the cart 140 may be connected to the processing device 120 through the network 150, or connected to the processing device 120 directly as illustrated by the bidirectional dotted arrow connecting the cart 140 and the processing device 120 in FIG. 1. As still another example, the storage device 130 may be connected to the medical device 110 through the network 150, or connected to the medical device 110 directly as illustrated by the bidirectional dotted arrow connecting the medical device 110 and the storage device 130 in FIG. 1. As still another example, the storage device 130 may be connected to the cart 140 through the network 150, or connected to the cart 140 directly as illustrated by the bidirectional dotted arrow connecting the cart 140 and the storage device 130 in FIG. 1.

The medical device 110 may be configured to acquire image data relating to a subject. The image data relating to a subject may include an image (e.g., an image slice), projection data, or a combination thereof. In some embodiments, the image data may be a two-dimensional (2D) image data, a three-dimensional (3D) image data, a four-dimensional (4D) image data, or the like, or any combination thereof. The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

In some embodiments, the medical device 110 may include a single modality imaging device. For example, the medical device 110 may include a single-photon emission computed tomography (SPECT) device, a positron emission tomography (PET) device, etc. In some embodiments, the medical device 110 may include a multi-modality imaging device. Exemplary multi-modality imaging devices may include a SPECT-CT device, a SPECT-PET device, a SPECT-MR device, etc. The medical device 110 may include a medical scanner 111 and a bed 112. A SPECT device may be taken as an example of the medical device 110, and not intended to limit the scope of the present disclosure. The medical scanner 111 of the SPECT device may include a gantry, a collimator, a detector, an electronics module, and/or other components not shown. The gantry may support one or more parts of the SPECT device, for example, the collimator, the detector, the electronics module, and/or other components. The collimator may collimate photons (e.g., y photons) emitted from an object being examined. The detector may be configured to detect the photons collimated by the collimator and/or generate electrical signals. The electronics module may collect and/or process electrical signals (e.g., scintillation pulses) generated by the detector. The electronics module may convert an analog signal (e.g., an electrical signal generated by the detector) relating to a photon detected by the detector to a digital signal to generate projection data. In some embodiments, the electronics module may be part of the detector. The bed 112 may be configured to support the object. In some embodiments, the bed 112 may move the object along a direction (e.g., Z direction shown in FIG. 1), so that the bed 112 may move into or out of the gantry of the medical device 110.

The processing device 120 may process data and/or information obtained from the medical device 110, the storage device 130, and/or the cart 140. For example, the processing device 120 may obtain an installation instruction for installing a target collimator into the medical scanner 111. As another example, the processing device 120 may identify, from a plurality of collimators stored in the tool storage device 160, the target collimator based on the installation instruction. As still another example, the processing device 120 may use the cart 140 to transport the target collimator from the tool storage device 160 to the medical scanner 111, and automatically install, using the cart, the target collimator into the medical scanner 111.

In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, and/or the cart 140 via the network 150. As another example, the processing device 120 may be directly connected to the medical device 110, the cart 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the cart 140. In some embodiments, the processing device 120 may be part of the medical device 110.

The storage device 130 may store data, instructions, and/or any other information. In some embodiments, the storage device 130 may store data and/or instructions that the processing device 120, and/or the cart 140 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storages may include a magnetic disk, an optical disk, a solid-state drive, etc. Exemplary removable storages may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the automatic installation system 100 (e.g., the processing device 120, the cart 140, etc.). One or more components in the automatic installation system 100 may access the data or instructions stored in the storage device 130 via the network 150. In some embodiments, the storage device 130 may be integrated into the medical device 110 or the cart 140.

The cart 140 may be connected to and/or communicate with the medical device 110, the processing device 120, the storage device 130, and/or the tool storage device 160. In some embodiments, the cart 140 may include a cart body, a mechanical arm, and wheels. In some embodiments, the cart 140 may include a mechanical arm, a cart body, wheels, a storage device, and a processor. The storage device may include a set of instructions, and when executing the set of instructions, the processor is configured to direct the cart to perform operations. The operations may include obtaining an installation instruction for installing a target collimator into a medical scanner; identifying, from a plurality of collimators stored in a tool storage device, the target collimator based on the installation instruction; griping, using the mechanical arm, the target collimator from the tool storage device to the cart body; controlling the cart body to transport the target collimator from the tool storage device to the medical scanner; and automatically installing, using the mechanical arm, the target collimator into the medical scanner. In some embodiments, the cart body may be configured to support a collimator (e.g., a target collimator or an installed collimator) for transport between the medical device 110 and the tool storage device 160. For example, the cart body may include a collimator platform and a driving mechanism mounted on the collimator platform. The collimator platform may be configured to support the collimator (e.g., the target collimator or the installed collimator). In some embodiments, the driving mechanism may be configured to communicate with the processing device 120 (or the processor) to receive instructions (e.g., installation instructions, uninstallation instructions) and control the mechanical arm to transport, install, or uninstall the collimator (e.g., the target collimator or the installed collimator). In some embodiments, the driving mechanism may be configured to communicate with the processing device 120 (or the processor) to receive instructions (e.g., installation instructions, uninstallation instructions) and control the collimator platform to move to change a relative position between the cart 140 and the tool storage device 160.

In some embodiments, the mechanical arm may include a gripper, a retractable arm, and a sensor. The gripper may be mounted on an end of the retractable arm and configured to grip the collimator (e.g., the target collimator or the installed collimator) during the transport of the collimator. The retractable arm may be configured to stretch and/or contract, and facilitate the transport of the collimator. The sensor may be configured to determine a state of the retractable arm and/or the gripper. For example, the sensor may include an image sensor for capturing an image of the gripper to determine whether the retractable arm moves the gripper to a predetermined location. As another example, the sensor may include a pressure sensor to determine whether the mechanical arm (e.g., the gripper) grips the collimator. The gripper, the retractable arm, and/or the sensor may be configured to communicate with the processing device 120 to receive instructions (e.g., griping instructions, stretching and/or contracting instructions) and control the mechanical arm to transport, install, or uninstall the collimator (e.g., the target collimator or the installed collimator).

The network 150 may include any suitable network that can facilitate the exchange of information and/or data for the automatic installation system 100. In some embodiments, one or more components of the automatic installation system 100 (e.g., the medical device 110, the processing device 120, the storage device 130, the cart 140, the terminal(s) 170, etc.) may communicate information and/or data with one or more other components of the automatic installation system 100 via the network 150. For example, the processing device 120 may obtain an installation instruction or an uninstallation instruction from the terminal(s) 170 via the network 150. As another example, the processing device 120 may instruct the cart 140 to perform one or more operations via the network 150. The network 150 may be and/or include a public network (e.g., the Internet), a private network (e.g., a local area network (LAN), a wide area network (WAN), etc.), a wired network (e.g., an Ethernet network), a wireless network (e.g., a Wi-Fi network), a cellular network (e.g., a Long Term Evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the automatic installation system 100 may be connected to the network 150 to exchange data and/or information.

The tool storage device 160 may be configured to store a plurality of collimators and/or QC tools. In some embodiments, the tool storage device 160 may include a plurality of storage spaces each of which is configured to store a collimator of the plurality of collimators. In some embodiments, the tool storage device 160 and the medical device 110 may be away from each other. As used herein, the words “away from” refers that the tool storage device 160 and the medical device 110 are two separate devices, and a distance between the two devices is greater than a distance threshold (e.g., 3 meters). In some embodiments, the tool storage device 160 may store a large number of collimators. Foe example, a count of the plurality of collimators stored in the tool storage device 160 may be greater than a count threshold (e.g., 3, 5, etc.).

The terminal(s) 170 may be connected to and/or communicate with the medical device 110, the processing device 120, the storage device 130, and/or the cart 140. In some embodiments, the terminal 170 may include a mobile device 171, a tablet computer 172, a laptop computer 173, or the like, or any combination thereof. For example, the mobile device 171 may include a mobile phone, a personal digital assistant (PDA), a gaming device, a navigation device, a point of sale (POS) device, a laptop, a tablet computer, a desktop, or the like, or any combination thereof. In some embodiments, the terminal 170 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the automatic installation system 100 may include one or more additional components and/or one or more components of the automatic installation system 100 described above may be omitted. Additionally or alternatively, two or more components of the automatic installation system 100 may be integrated into a single component. A component of the automatic installation system 100 may be implemented on two or more sub-components.

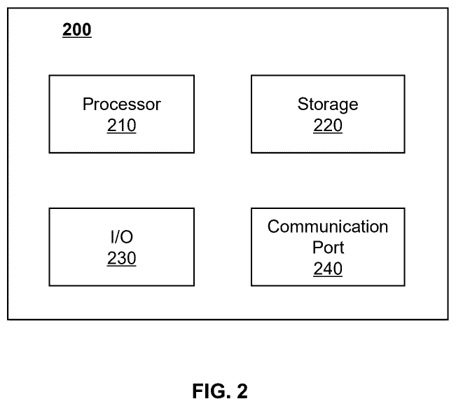

FIG. 2 is a schematic diagram illustrating exemplary hardware and/or software components of an exemplary computing device on which the processing device 120 may be implemented according to some embodiments of the present disclosure. As illustrated in FIG. 2, a computing device 200 may include a processor 210, a storage 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage 220 may store data/information obtained from the medical device 110, the cart 140, the storage device 130, and/or any other component of the automatic installation system 100. The storage 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the cart 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

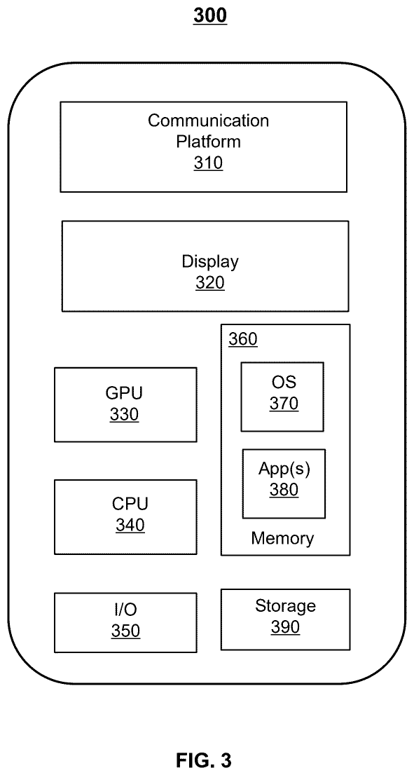

FIG. 3 is a schematic diagram illustrating exemplary hardware and/or software components of an exemplary mobile device according to some embodiments of the present disclosure. In some embodiments, the cart 140, the processing device 120, and/or the terminal(s) 170 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the automatic installation system 100, and enable data and/or signal to be transmitted between the mobile device 300 and other components of the automatic installation system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the automatic installation system 100. For example, the communication platform 310 may transmit data and/or signals inputted by a user to other components of the automatic installation system 100. The inputted data and/or signals may include a user instruction. As another example, the communication platform 310 may receive data and/or signals transmitted from the processing device 120. The received data and/or signals may include image data acquired by the medical device 110.

In some embodiments, a mobile operating system (OS) 370 (e.g., iOS™, Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information from the processing device 120. User interactions with the information stream may be achieved via the I/O 350 and provided to the processing device 120 and/or other components of the automatic installation system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

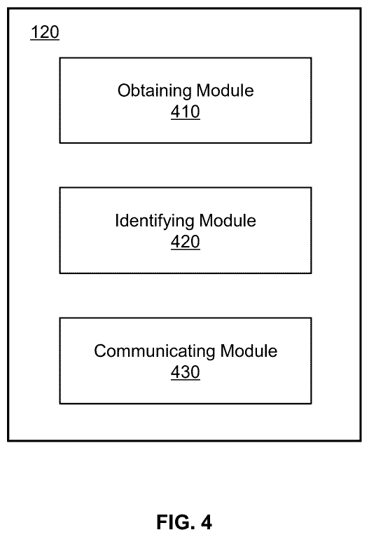

FIG. 4 is a block diagram illustrating an exemplary processing device according to some embodiments of the present disclosure. In some embodiments, the processing device 120 may include an obtaining module 410, an identifying module 420, and a control module 430.

The obtaining module 410 may be configured to obtain information. For example, the obtaining module 410 may obtain an instruction (e.g., an installation instruction, an uninstallation instruction, etc.). As another example, the obtaining module 410 may obtain a scan protocol.

The identifying module 420 may be configured to identify, from a plurality of collimators stored in the tool storage device 160, a target collimator based on the installation instruction.

The control module 430 may be configured to control the cart 140 to transport, install, or uninstall the target collimator. For example, the control module 430 may control the cart 140 to transport the target collimator from the tool storage device 160 to the medical scanner 111. As another example, the control module 430 may control the cart 140 to align, based on a navigation algorithm, the target collimator with a detector of the medical scanner 111. As still another example, the control module 430 may control the cart 140 to automatically install the target collimator into the medical scanner 111. As still another example, the control module 430 may control the cart 140 to automatically uninstall, using the cart 140, an installed collimator from the medical scanner 111 based on the installation instruction.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be added or omitted in the processing device 120. For example, the processing device 120 may further include a storage module (not shown in FIG. 4) configured to store data and/or information associated with the automatic installation system 100. In some embodiments, two or more modules may be integrated into a single module. For example, the obtaining module 410 and the control module 430 may be integrated into a single module.

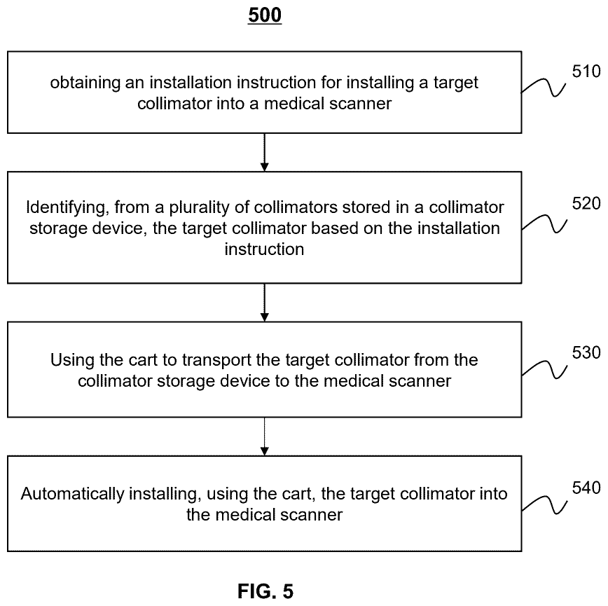

FIG. 5 is a flowchart illustrating an exemplary process 500 for automatically installing a target collimator according to some embodiments of the present disclosure. In some embodiments, the process 500 may be implemented in the automatic installation system 100 illustrated in FIG. 1. For example, the process 500 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 500 may be accomplished with one or more additional operations not described, and/or without one or more of the operations discussed. Additionally, the order in which the operations of the process 500 as illustrated in FIG. 5 and described below is not intended to be limiting.

In 510, the processing device 120 (e.g., the obtaining module 410) may obtain an installation instruction for installing a target collimator into a medical scanner (e.g., the medical scanner 111).

In some embodiments, the installation instruction may be directly sent from an operator by a terminal device (e.g., the terminal(s) 170, the medical device 110, etc.). The installation instruction may include a target identifier of the target collimator to be installed or an identifier of an installed collimator to be uninstalled, a collimator mode of the target collimator, an energy (e.g., a high energy, a low energy, etc.) of the target collimator, or the like, or any combination thereof. In some embodiments, the collimator mode may include a pinhole collimator, a parallel hole collimator, a fan-beam collimator, a cone-beam collimator, a slit-slat collimator, a conical hole collimator, or the like, or any combination thereof.

In some embodiments, the installation instruction may be determined based on scan protocol. For example, before scanning a subject (e.g., a patient), a scan protocol may be determined. For example, if the scan area of the subject is the chest, a scan protocol corresponding to a chest examination may be obtained. Further, the processing device 120 may determine the installation instruction based on the scan protocol of the subject. In some embodiments, the scan protocol may be previously generated (e.g., manually input by a user or determined by the processing device 120) and stored in a storage device (e.g., the storage device 130). The processing device 120 may retrieve the scan protocol from the storage device, and determine the installation instruction based on the scan protocol. In some embodiments, the scan protocol may be determined based on a contrast agent and/or an activity thereof, a radioactive tracer and/or an activity thereof, a required image quality (e.g., an image resolution, an image signal-to-noise ratio, a sensitivity, etc.), a selection of an operator (e.g., a scan duration, a field of view (FOV), etc.), etc. For example, a size (e.g., a hole size) of the target collimator may be determined based on a required image quality (e.g., an image resolution, a sensitivity, etc.). A collimator mode of the target collimator may be determined based on FOV. An energy (e.g., a high energy, a low energy, etc.) of the target collimator may be determined based on radioactive tracer.

The scan protocol may include, for example, value(s) or value range(s) of scan parameter(s), a portion of the subject to be scanned, feature information of the subject (e.g., the gender, the body shape), a collimator mode of the target collimator to be installed and/or a collimator mode of an installed collimator to be uninstalled, a target identifier of the target collimator to be installed and/or an identifier of an installed collimator to be uninstalled, or the like, or any combination thereof. In some embodiments, the scan parameter(s) may include a scanning mode, a bed position, a voltage of a radiation source, a current of the radiation source, a distance between the radiation source and a detector (also referred to as a source image distance, or a SID), a radiation dose, a scan time, a field of view (FOV), whether to record physiological parameters (e.g., electrocardio (ECG) signal, etc.) synchronously, or the like, or any combination thereof. Merely by way of example, a scan protocol of a myocardial perfusion scanning may include a scanning time of 20 minutes, a single bed, a synchronous recording of ECG signals, a low-energy conical hole collimator, etc. Based on the scan protocol of the myocardial perfusion scanning, the installation instruction may be determined. Correspondingly, the installation instruction may include a conical hole collimator with low energy. More descriptions regarding determining the installation instruction based on the scan protocol may be found elsewhere in the present disclosure. See, e.g., FIG. 12 and the descriptions thereof. In some embodiments, the processing device 120 may send the installation instruction to the cart 140 via the network 150 (e.g., wireless local area network or Bluetooth, etc.). The cart 140 may receive the installation instruction from the processing device 120 and perform an installation process according to the installation instruction.

In 520, the processing device 120 (e.g., the identifying module 420) may identify, from a plurality of collimators stored in the tool storage device 160, the target collimator based on the installation instruction.

In some embodiments, the tool storage device 160 may include a plurality of collimators. The plurality of collimators may include different modes of collimators, different energies of collimators, etc. In some embodiments, each collimator may be labeled with an identifier. The identifier may include a mechanical switch, a proximity switch, a photoelectric switch, an electromagnetic sensor, a radiofrequency identification tag, a unique symbol (e.g., a QR code, a bar code, etc.), or the like, or any combination thereof.

In some embodiments, the processing device 120 may identify, from the plurality of collimators stored in the tool storage device 160, the target collimator based on the installation instruction. For example, the processing device 120 may obtain a target identifier of the target collimator based on the installation instruction. As described above, the installation instruction may include a target identifier of the target collimator to be installed. The processing device 120 may obtain the target identifier of the target collimator from the installation instruction. The processing device 120 may identify a plurality of identifiers respectively corresponding to the plurality of collimators stored in the tool storage device 160. In some embodiments, the processing device 120 may identify the target collimator based on the plurality of identifiers respectively corresponding to the plurality of collimators. For example, the processing device 120 may identify the target collimator by matching the target identifier with one of the plurality of identifiers respectively corresponding to the plurality of collimators. More descriptions regarding identifying the target collimator may be found elsewhere in the present disclosure. See, e.g., FIG. 10 and the descriptions thereof.

In 530, the processing device 120 (e.g., the control module 430) may use the cart 140 to transport the target collimator from the tool storage device 160 to the medical scanner 111.

In some embodiments, the processing device 120 may use the cart 140 to grip the target collimator from the tool storage device 160 and support the target collimator. For example, the mechanical arm (e.g., the gripper) of the cart 140 may grip the target collimator from the tool storage device 160 after identifying the target collimator. In some embodiments, the processing device 120 may transport, using the retractable arm of the cart 140, the target collimator to the cart body. For example, the processing device 120 may control the gripper to grip the target collimator, and control the retractable arm to extend or retract to transport the target collimator to the cart body. In some embodiments, the cart body (e.g., the collimator platform) may support the target collimator during a transportation process of the target collimator from the tool storage device 160 to the medical scanner 111.

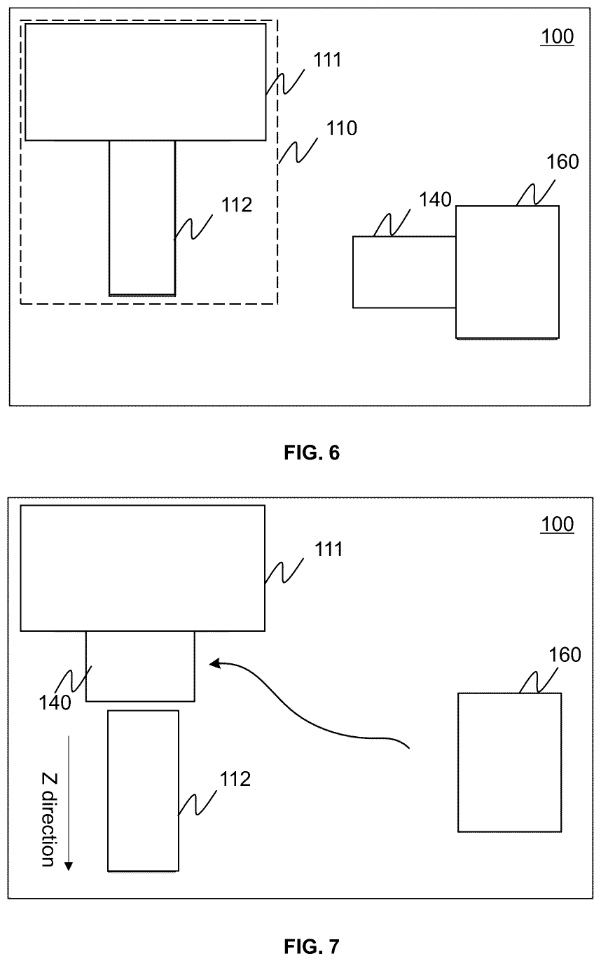

In some embodiments, to facilitate the automatic installation of the target collimator, the processing device 120 may use the cart 140 (e.g., the wheels of the cart 140) to transport the target collimator from the tool storage device 160 to a predetermined location of the medical scanner 111. For example, the predetermined location may be close to a detector of the medical scanner 111. FIG. 6 is a schematic diagram illustrating an exemplary automatic installation system 100 according to some embodiments of the present disclosure. As shown in FIG. 6, the medical device 110 may include the medical scanner 111 and the bed 112. During a scanning process of an object, the bed 112 may be configured to support the object and may be close to the medical scanner 111. As shown in FIG. 6, the cart 140 may be close to the tool storage device 160 to grip the target collimator from the tool storage device 160. In some embodiments, the tool storage device 160 and the medical device 110 may be away from each other. As used herein, the words “away from” refers that the tool storage device 160 and the medical device 110 are two separate devices, and a distance between the two devices is greater than a distance threshold (e.g., 3 meters, 5 meters, etc.). In some embodiments, the tool storage device 160 and the medical device 110 may be in a same scanning room (e.g., an examination room of a hospital), and the cart 140 may transport the target collimator between the tool storage device 160 and the medical device 110 in the same scanning room. In some embodiments, the tool storage device 160 and the medical device 110 may be in separate rooms (e.g., different rooms of a hospital), and the cart 140 may transport the target collimator between the tool storage device 160 and the medical device 110 in the hospital. For example, the tool storage device 160 may store collimators of a plurality of medical devices. The cart 140 may transport a collimator (e.g., the target collimator) from the tool storage device 160 to any one of the medical devices.

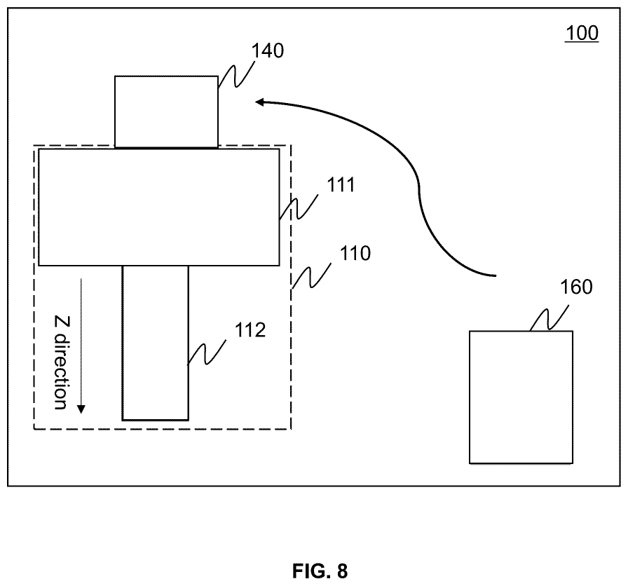

FIG. 7 is a schematic diagram illustrating an exemplary automatic installation system 100 according to some embodiments of the present disclosure. FIG. 8 is a schematic diagram illustrating an exemplary automatic installation system 100 according to some embodiments of the present disclosure. As shown in FIG. 7, the medical scanner 111 and/or the bed 112 may move relative to each other. After the cart 140 griping the target collimator (as shown in FIG. 6), the bed 112 may move along the direction (e.g., Z direction shown in FIG. 7) in which the bed 112 may move into or out of the gantry of the medical device 111. The cart 140 may transport the target collimator from the tool storage device 160 to a predetermined location of the medical scanner 111. As shown in FIG. 7, the predetermined location may include a location between the medical scanner 111 and the bed 112 and the predetermined location may be close to the medical scanner 111. Alternatively, as shown in FIG. 8, after the cart 140 griping the target collimator (as shown in FIG. 6), the cart 140 may transport the target collimator from the tool storage device 160 to a location close to a side of the medical scanner 111 that is away from the bed 112 along the direction (e.g., Z direction shown in FIG. 8) in which the bed 112 may move into or out of the gantry of the medical device 111.

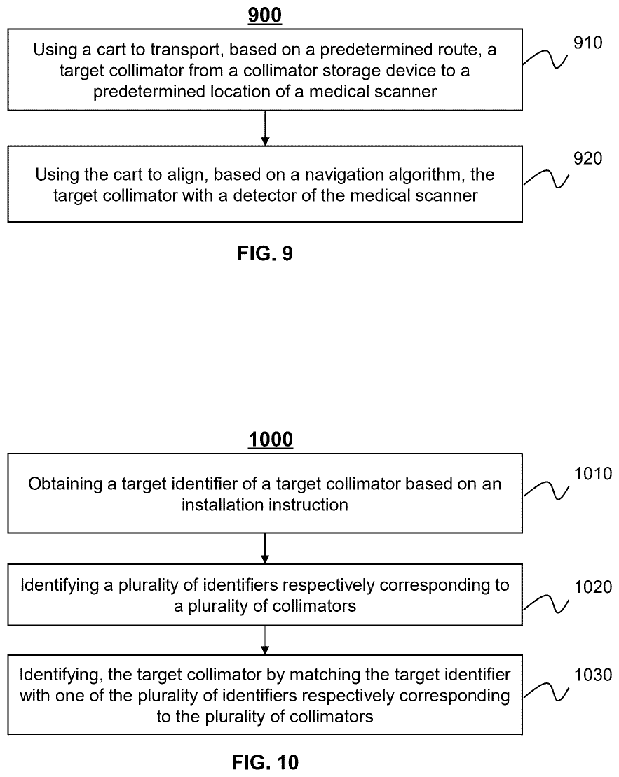

In some embodiments, the processing device 120 may use the cart 140 to transport, based on a predetermined route, the target collimator from the tool storage device 160 to the predetermined location of the medical scanner 111. In some embodiments, the processing device 120 may use the cart 140 to align, based on a navigation algorithm, the target collimator with a detector of the medical scanner 111. For example, the cart 140 may align the target collimator with a fixture of the detector to facilitate the automatic installation of the target collimator. In some embodiments, an alignment accuracy of the alignment based on the navigation algorithm may be millimeter level. More descriptions regarding transporting and aligning the target collimator may be found elsewhere in the present disclosure. Sec, e.g., FIG. 9 and the descriptions thereof.

In 540, the processing device 120 (e.g., the control module 430) may automatically install, using the cart 140, the target collimator into the medical scanner 111.

As used herein, the word “automatically” may refer that the installation of the target collimator is performed in a mechanical manner without any human assistance. In some embodiments, the processing device 120 may automatically install, by a locating module of the cart 140, the target collimator onto a detector of the medical scanner 111. In some embodiments, the locating module may include a locating hole, a locating pin, a bolt, a nut, or the like, or any combination thereof. In some embodiments, a locating accuracy of the locating module may be 0.25 millimeter level. For example, the locating module of the cart 140 may include a locating hole, and the medical scanner 111 may include a locating pin that is paired with the locating hole. As another example, the locating module of the cart may include a locating pin, and the medical scanner 111 may include a locating hole that is paired with the locating pin. The locating hole may match with the locating pin, and the processing device 120 may control the gripper of the cart 140 to mount the target collimator onto the medical scanner 111. In some embodiments, the target collimator may be installed by fixedly connecting with the detector of the medical scanner 111.

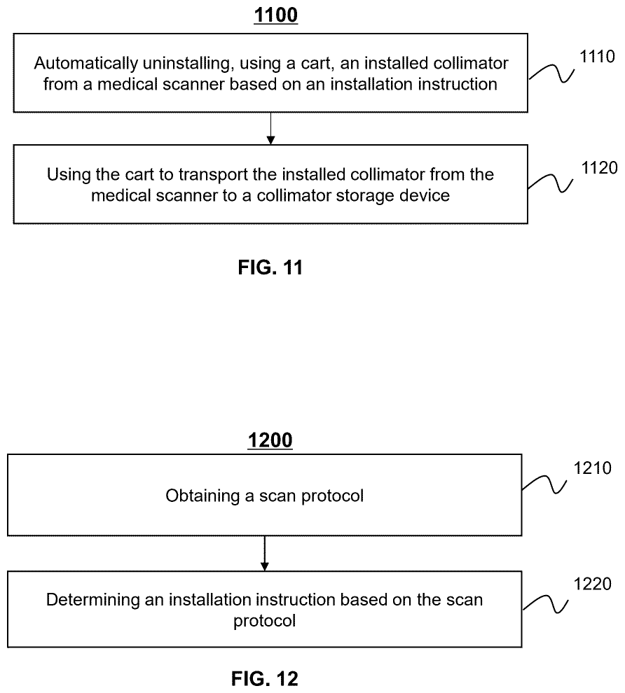

In some embodiments, before installing the target collimator, the medical scanner 111 may include an installed collimator that is pre-installed thereon. The processing device 120 may automatically uninstall the installed collimator before installing the target collimator. For example, the processing device 120 may automatically uninstall, using the cart 140, the installed collimator from the medical scanner 111 based on the installation instruction. For example, after the cart 140 transports the target collimator to the predetermined location of the medical scanner 111 and before installing the target collimator, the processing device 120 may control the cart 140 to uninstall the installed collimator. For example, the gripper of the cart 140 may grip the installed collimator and transport the installed collimator from the medical scanner 111 to the cart 140 (e.g., the installed collimator may be transported to the collimator platform of the cart 140). In some embodiments, the processing device 120 may use the cart 140 to transport the installed collimator from the medical scanner 111 to the tool storage device 160. For example, after uninstalling the installed collimator and installing the target collimator, the cart 140 may transport the installed collimator to the tool storage device 160. In some embodiments, the installation of the target collimator and the uninstallation of the installed collimator may be performed by a same cart 140. Alternative, the installation of the target collimator and the uninstallation of the installed collimator may be performed by two different carts 140. For example, one cart may be configured to install the target collimator, and the other cart may be configured to uninstall the installed collimator. More descriptions regarding uninstalling the installed collimator may be found elsewhere in the present disclosure. See, e.g., FIG. 11 and the descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, one or more operations may be added into the process 500. For example, before operation 540 for automatically installing the target collimator, an uninstallation process of the installed collimator may be performed.

FIG. 9 is a flowchart illustrating an exemplary process 900 for transporting a target collimator according to some embodiments of the present disclosure. In some embodiments, the process 900 may be implemented in the automatic installation system 100 illustrated in FIG. 1. For example, the process 900 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 900 may be accomplished with one or more additional operations not described, and/or without one or more of the operations discussed. Additionally, the order in which the operations of the process 900 as illustrated in FIG. 9 and described below is not intended to be limiting. In some embodiments, operation 530 may be performed according to process 900.

In 910, the processing device 120 (e.g., the control module 430) may use the cart 140 to transport, based on a predetermined route, the target collimator from the tool storage device 160 to a predetermined location of the medical scanner 111.

In some embodiments, the tool storage device 160 and the medical scanner 111 may be located at two relative fixed positions. The predetermined route between the tool storage device 160 and the medical scanner 111 may be predetermined and stored in a storage device (e.g., the storage device 130) or the cart 140. For example, the predetermined route may be determined by an operator of the automatic installation system 100 and transmit to the storage device or the cart 140. As another example, the predetermined route may be determined by the processing device 120 based on an indoor wireless positioning technologies. Exemplary indoor wireless positioning technologies may include Wi-Fi positioning technology, Bluetooth positioning technology, infrared positioning technology, ultra-wideband positioning technology, RFID positioning technology, ZigBee positioning technology, motion capture positioning technology, ultrasonic positioning technology, or the like, or any combination thereof. In some embodiments, the processing device 120 may retrieve the predetermined route from the storage device, and transport, based on the predetermined route, the target collimator from the tool storage device 160 to the predetermined location of the medical scanner 111. In some embodiments, the cart 140 may store the predetermined route, and once obtaining an installation instruction, the cart 140 may directly transport the target collimator based on the predetermined route to improve an efficiency for obtaining the predetermined route. In some embodiments, the predetermined route may be included in the installation instruction. The processing device 120 may parse the installation instruction and send the predetermined route to the cart 140. The cart 140 may obtain the predetermined route to transport the target collimator. In some embodiments, the predetermined location may be a location that facilitate the installation of the target collimator. More descriptions regarding the predetermined location may be found elsewhere in the present disclosure. See, e.g., FIGS. 5, 7, and 8 and the descriptions thereof.

In 920, the processing device 120 (e.g., the control module 430) may use the cart 140 to align, based on a navigation algorithm, the target collimator with a detector of the medical scanner 111.

In some embodiment, an alignment accuracy of the alignment based on the navigation algorithm may be millimeter level. Exemplary navigation algorithm may include a laser navigation algorithm, a visual navigation algorithm, an inertial navigation algorithm, or the like, or any combination thereof. In some embodiments, the navigation algorithm may be stored in a storage device (e.g., the storage device 130), and the processing device 120 may retrieve the navigation algorithm from the storage device, and control the cart 140 to align the target collimator based on the navigation algorithm. In some embodiments, the cart 140 may align the target collimator with a fixture (e.g., a groove, a pin, etc.) of the detector to facilitate the automatic installation of the target collimator. For example, the cart 140 may include an image sensor for capturing an image of the detector of the medical scanner 111. The cart 140 may align the target collimator with the detector based on the image captured from the image sensor.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 10 is a flowchart illustrating an exemplary process 1000 for identifying a target collimator according to some embodiments of the present disclosure. In some embodiments, the process 1000 may be implemented in the automatic installation system 100 illustrated in FIG. 1. For example, the process 1000 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1000 may be accomplished with one or more additional operations not described, and/or without one or more of the operations discussed. Additionally, the order in which the operations of the process 1000 as illustrated in FIG. 10 and described below is not intended to be limiting. In some embodiments, operation 520 may be performed according to process 1000.

In 1010, the processing device 120 (e.g., the identifying module 420) may obtain a target identifier of the target collimator based on the installation instruction. In some embodiments, the installation instruction may include a target identifier of the target collimator to be installed, and the processing device 120 may parse the installation instruction to obtain the target identifier of the target collimator.

In 1020, the processing device 120 (e.g., the identifying module 420) may identify a plurality of identifiers respectively corresponding to the plurality of collimators.

In some embodiments, each of the plurality of collimators stored in the tool storage device 160 may be labeled with an identifier. The identifier may include a mechanical switch (e.g., a contact switch), a proximity switch, a photoelectric switch (e.g., a photoelectric correlated cell), an electromagnetic sensor (e.g., a Hall element), a radiofrequency identification tag, a unique symbol (e.g., a QR code, a bar code, etc.), or the like, or any combination thereof. For example, each of the plurality of collimators may be labeled with a QR code. The cart 140 (e.g., a sensor of the cart 140) may scan the QR codes of the plurality of collimators to identify the plurality of identifiers respectively corresponding to the plurality of collimators. In some embodiments, each of the plurality of collimators may be stored at a fixed storage space of the tool storage device 160. Once the cart 140 finishes scanning the QR codes of the plurality of collimators, a mapping relationship between a QR code of a collimator and a corresponding storage space that stores the collimator may be stored in a storage device (e.g., the storage device 130). Each time the processing device 120 obtains an installation instruction, the processing device 120 may access the storage device to obtain a storage space that stores a required collimator. The cart 140 may grip the required collimator from the storage space of the tool storage device 160. In this way, scanning all of the QR codes of the plurality of collimators each time when the processing device 120 obtains an installation instruction may be avoided, thereby saving an identify time for identifying the collimators.

In 1030, the processing device 120 (e.g., the identifying module 420) may identify the target collimator by matching the target identifier with one of the plurality of identifiers respectively corresponding to the plurality of collimators.

In some embodiments, the processing device 120 may match the target identifier with one of the plurality of identifiers respectively corresponding to the plurality of collimators. For example, a mechanical switch (e.g., a contact switch) may be triggered by the processing device 120, and a collimator being connected to the mechanical switch may be identified as the target collimator. As another example, the target collimator may be labeled with a target QR code. The processing device 120 may scan QR codes of the plurality of collimators, and identify a collimator with a QR code that is same with the target QR code as the target collimator. In some embodiments, if there is no identifier matching with the target identifier, the processing device 120 may generate a notice indicating that there is no target collimator in the tool storage device 160.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 11 is a flowchart illustrating an exemplary process 1100 for uninstalling an installed collimator according to some embodiments of the present disclosure. In some embodiments, the process 1100 may be implemented in the automatic installation system 100 illustrated in FIG. 1. For example, the process 1100 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1100 may be accomplished with one or more additional operations not described, and/or without one or more of the operations discussed. Additionally, the order in which the operations of the process 1100 as illustrated in FIG. 11 and described below is not intended to be limiting.

In 1110, the processing device 120 (e.g., the control module 430) may automatically uninstall, using the cart 140, an installed collimator from the medical scanner based on the installation instruction.

In some embodiments, before installing the target collimator, the medical scanner 111 may include an installed collimator that is pre-installed thereon. The processing device 120 may automatically uninstall the installed collimator before installing the target collimator. For example, the processing device 120 may automatically uninstall, using the cart 140, the installed collimator from the medical scanner 111 based on the installation instruction. For example, after the cart 140 transports the target collimator to the predetermined location of the medical scanner 111 and before installing the target collimator, the processing device 120 may control the cart 140 to uninstall the installed collimator. For example, the gripper of the cart 140 may grip the installed collimator and transport the installed collimator from the medical scanner 111 to the cart 140. For example, the installed collimator may be transported to the collimator platform of the cart 140.

In some embodiments, the installation instruction may include determining whether there is an installed collimator pre-installed on the medical scanner 111. In response to determining that there is an installed collimator that is pre-installed on the medical scanner 111, the processing device 120 may control the cart 140 to uninstall the installed collimator before installing the target collimator. In response to determining that there is not an installed collimator that is pre-installed on the medical scanner 111, the processing device 120 may control the cart 140 to directly install the target collimator. In some embodiments, the cart 140 may include a sensor (e.g., an image sensor) to determine whether there is an installed collimator.

In 1120, the processing device 120 (e.g., the control module 430) may use the cart 140 to transport the installed collimator from the medical scanner 111 to the tool storage device 160.

In some embodiments, the processing device 120 may use the cart 140 to transport the installed collimator from the medical scanner 111 to the tool storage device 160. For example, after uninstalling the installed collimator and installing the target collimator, the cart 140 may transport the installed collimator to the tool storage device 160. In some embodiments, the processing device 120 may identify the installed collimator to obtain an identifier of the installed collimator, and control the cart 140 to store the installed collimator into the tool storage device 160. For example, the processing device 120 may access the storage device (e.g., the storage device 130) to obtain a mapping relationship between the identifiers of the plurality of collimators and the storage spaces. The processing device 120 may control the cart 140 to store the installed collimator into a corresponding storage space of the tool storage device 160 based on the mapping relationship.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 12 is a flowchart illustrating an exemplary process 1200 for determining an installation instruction according to some embodiments of the present disclosure. In some embodiments, the process 1200 may be implemented in the automatic installation system 100 illustrated in FIG. 1. For example, the process 1200 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1200 may be accomplished with one or more additional operations not described, and/or without one or more of the operations discussed. Additionally, the order in which the operations of the process 1200 as illustrated in FIG. 12 and described below is not intended to be limiting. In some embodiments, operation 510 may be performed according to process 1200.

In 1210, the processing device 120 (e.g., the obtaining module 410) may obtain a scan protocol.

In some embodiments, the scan protocol may include, for example, value(s) or value range(s) of scan parameter(s), a portion of the subject to be scanned, feature information of the subject (e.g., the gender, the body shape), a collimator mode of the target collimator to be installed and/or a collimator mode of an installed collimator to be uninstalled, a target identifier of the target collimator to be installed and/or an identifier of an installed collimator to be uninstalled, or the like, or any combination thereof. In some embodiments, the scan parameter(s) may include a scanning mode, a bed position, a voltage of a radiation source, a current of the radiation source, a distance between the radiation source and a detector (also referred to as a source image distance, or a SID), a radiation dose, a scan time, a field of view (FOV), whether to record physiological parameters (e.g., electrocardio (ECG) signal, etc.) synchronously, or the like, or any combination thereof. In some embodiments, the processing device 120 may obtain the scan protocol from an input of an operator of the automatic installation system 100. In some embodiments, the processing device 120 may obtain scan parameter(s) input by the operator of the automatic installation system 100, and generate the scan protocol based on the scan parameter(s).

In some embodiments, the scan protocol may be previously generated (e.g., manually input by a user or determined by the processing device 120) and stored in a storage device (e.g., the storage device 130). The processing device 120 may retrieve the scan protocol from the storage device. In some embodiments, the scan protocol may be determined based on a contrast agent and/or an activity thereof, a radioactive tracer and/or an activity thereof, a required image quality (e.g., an image resolution, an image signal-to-noise ratio, a sensitivity, etc.), a selection of an operator (e.g., a scan duration, a field of view (FOV), etc.), etc. For example, a size (e.g., a hole size) of the target collimator may be determined based on a required image quality (e.g., an image resolution, a sensitivity, etc.). A collimator mode of the target collimator may be determined based on FOV. An energy (e.g., a high energy, a low energy, etc.) of the target collimator may be determined based on radioactive tracer.

In 1220, the processing device 120 (e.g., the obtaining module 410) may determine the installation instruction based on the scan protocol.

As described above, the scan protocol may include a collimator mode of the target collimator to be installed and/or a collimator mode of an installed collimator to be uninstalled, a target identifier of the target collimator to be installed and/or an identifier of an installed collimator to be uninstalled, etc. The processing device 120 may determine the installation instruction based on the scan protocol. For example, the installation instruction may include a target identifier of the target collimator to be installed or an identifier of an installed collimator to be uninstalled, a collimator mode of the target collimator, an energy (e.g., a high energy, a low energy, etc.) of the target collimator, or the like, or any combination thereof.

Merely by way of example, a scan protocol of a myocardial perfusion scanning may include a scanning time of 20 minutes, a single bed, a synchronous recording of ECG signals, a low-energy conical hole collimator, etc. Based on the scan protocol of the myocardial perfusion scanning, the installation instruction may be determined. Correspondingly, the installation instruction may include a conical hole collimator with low energy.

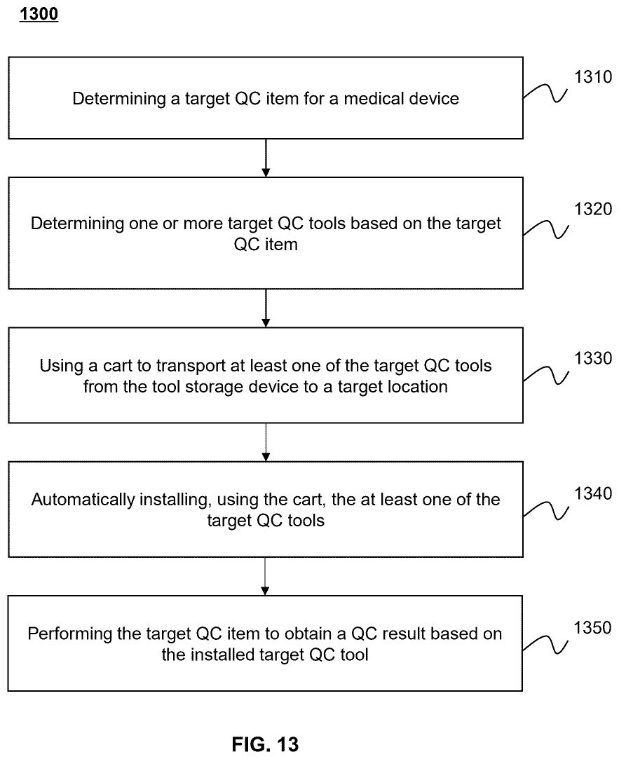

FIG. 13 is a flowchart illustrating an exemplary process for automatically quality control (QC) of a medical device according to some embodiments of the present disclosure. In some embodiments, the process 1300 may be implemented in the automatic installation system 100 illustrated in FIG. 1. For example, the process 1300 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1300 may be accomplished with one or more additional operations not described, and/or without one or more of the operations discussed. Additionally, the order in which the operations of the process 1300 as illustrated in FIG. 13 and described below is not intended to be limiting.

A medical device, such as an imaging device (e.g., a SPECT device) and a treatment device (e.g., a linear accelerator) may be used for diagnostic imaging and/or treatment of a subject. A traditional quality control (QC) system for a medical device typically requires multiple collimator changes or QC fixture replacements, a process often performed manually, which makes it cumbersome and time-consuming.

The present disclosure proposes an automated QC method that significantly improves the efficiency of quality control of a medical device, reducing labor and time costs, optimizing workflow, enhancing system stability and image quality, and increasing throughput for both clinical and preclinical applications.

QC of a medical device may include testing, monitoring, and/or maintenance procedures to ensure that the medical device is in an optimal working condition and meets the requirements of clinical diagnosis or treatment (e.g., meets a reference standard). In some embodiments, the QC of a medical device may include a test and/or a calibration of the medical device. The test may be used to verify whether the performance of the medical device meets a reference standard by performing one or more test items. The calibration can be used to ensure the performance of the medical device meets the reference standard by performing one or more calibration items.

In some embodiments, a calibration item may be associated with a test item, and the calibration item may be performed by adjusting one or more device parameters or compensating for systematic errors based on the result of the associated test item. A calibration item and a test item associated with the calibration item may correspond to the same QC index or be performed for the same performance of the medical device.

In some embodiments, a calibration item may be performed without the test item. For example, the calibration item may be performed after equipment installation or a major overhaul. As another example, the calibration item may be performed periodically. As still another example, a calibration item may be performed after one or more key components of the medical device are repaired or replaced.

In some embodiments, a test item may be performed after a calibration item is performed.

In 1310, the processing device 120 (e.g., the obtaining module 1710) may determine a target QC item for a medical device. As used herein, obtaining a target QC item refers to receiving information of the target QC item, such as the type of the target QC item.

A QC item refers to a specific content of QC, such as testing, calibration, verification, and/or optimization of a key parameter, an index, or an operational procedure to ensure equipment performance, safety, and imaging/therapeutic quality of the medical device. The target QC item refers to the QC item of the medical device that needs to be performed at the current time.

A QC item may include a test item (also referred to as a verification item) or a calibration item. The test item may be performed to verify whether the performance of the medical device meets a reference standard. The calibration item may be performed to ensure that the performance of the medical device meets the reference standard.

For example, if the medical device is a SPECT device, the test item (i.e., the type of the test item) may include a uniformity test, a spatial resolution test, a sensitivity test, an energy resolution test, an attenuation calibration verification, etc.; the calibration item (i.e., the type of the calibration item) may include an uniformity calibration, a sensitivity calibration, a spatial linearity calibration, an energy peak calibration, a center of rotation (COR) calibration, a multi-probe consistency calibration, an energy window width optimization, etc. As another example, if the medical device is a CT device, the test item may include a uniformity test, a noise level test, a spatial resolution test, a linearity and consistency test of CT values, etc.; the calibration item may include an air calibration, a detector gain calibration, a uniformity calibration, a CT value calibration, a detector response consistency calibration, a spectral calibration, etc. As still another example, if the medical device is an MRI device, the test item may include a homogeneity test, a spatial resolution test, a slice thickness test, a gradient linearity test, an SNR test, an artifact test, etc.; the calibration item may include an RF power calibration, a center frequency calibration, a gradient linearity calibration, a uniformity calibration of radio frequency field, etc. As still another example, if the medical device is a treatment device (e.g., a linear accelerator), the test item may include an output dose stability test, a beam flatness and symmetry test for radiation fields, etc.; the calibration item may include an absolute dose calibration, a relative dose calibration, a MLC position calibration, a gantry angle calibration, etc.

The target QC item may be determined by the processing device 120 based on operator input or according to historical QC data of the medical device. The historical QC data of the medical device may include a type of previous QC item (e.g., the last QC item), a result of the previous QC item, the time of the previous QC item, or the like, or a combination thereof.

For example, the target QC item may be input by the operator via the interface of a QC application implemented on the terminal device, and the processing device 120 may obtain the target QC item from the terminal device.

As another example, the processing device 120 may determine whether the target QC item needs to be performed based on the time when the previous target QC item was performed. If the processing device 120 determines that the current time has reached the QC cycle of the target QC item of the medical device (e.g., SPECT) based on the time when the previous target QC item was performed, the processing device 120 may determine that the target QC item needs to be performed at the current time. In response to determining that the target QC item needs to be performed at the current time, the processing device 120 may determine the target QC item.

As still another example, the processing device 120 may determine that the medical device is repaired before the current time, then the processing device 120 may determine the target QC item according to the repair of the medical device (e.g., a component of the medical device that is replaced). The repair data of the medical device may be obtained from the device log. Further, if the processing device 120 determines that a detector of the medical device is repaired, the processing device 120 may determine that the target QC item may include one or more test items and/or one or more calibration items associated with the detector (e.g., a uniformity test, a spatial resolution test, a sensitivity test, an energy resolution test, an uniformity calibration, a sensitivity calibration, a spatial linearity calibration, an energy peak calibration, a center of rotation (COR) calibration, a multi-probe consistency calibration, an energy window width optimization, etc.).

As still another example, the processing device 120 may determine whether the medical device is moved before the current time, then the processing device 120 may determine the target QC item according to the movement of the medical device.

As still another example, the processing device 120 may determine the target QC item based on the result or type of a previous QC item. If the previous QC item includes a test item, and the result of the test item indicates that the performance of the medical device does not meet a reference standard, the processing device 120 may determine that the target QC item includes a calibration item associated with the previous test item. If the previous QC item includes a calibration item, the processing device 120 may determine that the target QC item may include a test item associated with the calibration item, and the test item may be performed to verify whether the result of the previous calibration item is qualified.

In some embodiments, the processing device 120 may obtain a QC request (also referred to as a QC instruction) and determine the target QC item in response to receiving the QC request. A QC request refers to a trigger initiated when a QC procedure needs to be performed on a medical device (e.g., a SPECT device) via a QC application. Upon receiving the QC request or instruction, the processing device 120 may execute the corresponding target QC item to determine a QC result.

In some embodiments, the QC request may be sent from an operator via a terminal device (e.g., the terminal(s) 170, the medical device 110, etc.). The QC request may include information (e.g., the type) associated with the target QC item. The processing device 120 may determine the target QC item based on the QC request. The QC request or instruction may be triggered by the operator via the interface of the QC application implemented on the terminal device. The triggering manner may include a click by using an input device (e.g., mouse, keyboard), voice control, touchscreen interaction, etc.

In some embodiments, the QC request may be triggered automatically based on a predefined rule. The predefined rule may include a trigger condition of a QC request associated with a QC item. In some embodiments, the trigger condition of a QC request associated with a QC item may be determined based on a QC cycle of the QC item and if the current time reaches the QC cycle of the target QC item, the QC request associated with the target QC item may be generated and the processing device 120 may determine the target QC item based on the QC request. For example, when the processing device 120 detects that the current time has reached the QC cycle of the target QC item of the medical device (e.g., SPECT), the processing device 120 may automatically initiate the QC request according to the predefined rule and then determine the target QC item. In some embodiments, the trigger condition of a QC request associated with a QC item may be determined based on historical QC data (e.g., a type and/or result) of a previous QC item. If the previous QC item includes a test item, and the result of the test item indicates that the performance of the medical device does not meet a reference standard, the trigger condition may be satisfied, and the target QC item may be a calibration item associated with the previous QC item. If the previous QC item includes a calibration item, the trigger condition may be satisfied, and the target QC item may be a test item associated with the previous QC item, and the calibration item may be performed to verify whether the result of the previous calibration item is qualified.

In some embodiments, the processing device 120 may send the QC request to the cart 140 via the network 150 (e.g., wireless local area network or Bluetooth, etc.). The cart 140 may receive the QC request from the processing device 120. The cart 140 may determine the target QC item based on the QC request.

In 1320, the processing device 120 (e.g., the obtaining module 410) may determine one or more target QC tools based on the target QC item.

A QC tool refers to a component used for performing a QC item. The QC tool may include a radiation source, a phantom, a QC fixture, a collimator, testing software, or the like, or a combination thereof.

The radiation source may include a solid source, a liquid source contained in a vial or tube, or a radioactive source phantom, etc. The radiation source may include ⁹⁹Tc, ¹⁸F, ⁵⁷Co, ²²Na, etc. In some embodiments, the radiation source may be placed in a phantom. For example, the radiation source may be placed in a flood source planar phantom, a columnar phantom, a water phantom, etc. In some embodiments, the radiation source may include a point source, a line source, etc.

According to the shape of the phantom, the phantom may include a point source phantom, a line phantom, a lead grid phantom, a flood source planar phantom, a columnar phantom, etc. According to the function of the phantom, the phantom may include a water phantom, a dose phantom, an ACR (American college of Radiology) CT phantom, an ACR MRI phantom, a resolution phantom, a Jaszczak phantom, a Catphan phantom, etc. A phantom may include a radiation source or not include a radiation source.

Different QC items may correspond to different phantoms. For example, for the CT device, an ACR CT phantom may be used for the uniformity test; a dose phantom may be used for the dose test; a Catphan phantom may be used for the spatial resolution test. As another example, for the MRI device, an ACR MRI phantom may be used for the homogeneity test and the SNR test; a dynamic biomimetic phantom may be used for the time resolution test; a Magphan phantom may be used for the spatial distortion calibration. As still another example, for the SPECT device, a Jaszczak phantom may be used for the attenuation calibration verification; a line phantom may be used for the COR calibration.

As used herein, the radiation source and/or the phantom used in a QC item may also be referred to as a QC subject. The QC item may be performed to obtain data (e.g., image data) of the QC subject, analyze the data of the QC subject, and obtain a QC result.

The QC fixture may be a specialized tool for support, fixation, positioning, or auxiliary detection in the QC of the medical device. In some embodiments, a QC fixture may be used to position a radiation source or a phantom. In some embodiments, the QC fixture may include a phantom for accommodating a radiation source. For example, the QC fixture may include a floor source phantom for accommodating ⁹⁹Tc.

In some embodiments, the QC fixture may include an auxiliary detection device, such as a laser locator, a sensor, etc., for auxiliary positioning and calibration.

Different phantoms or radiation sources may correspond to different QC fixtures. For example, the QC fixture may include a graduated rotation mount used for placing the ACR CT phantom. As another example, the QC fixture may include an anti-skid mounting platform with central alignment ring for positioning the water phantom. As still another example, the QC fixture may include a precision guide rail and a point source locator for positioning the line phantom. As still another example, the QC fixture may include a lead shielding mount and an activity calibration well for positioning the Jaszczak phantom.

In some embodiments, specific QC items need to be performed based on a collimator, thus the collimator may also be a QC tool. For example, for the SPECT device, each of the spatial resolution test, the COR calibration, the sensitivity test, the uniformity test, etc., may need to be performed using a collimator. The collimator mode may include a pinhole collimator, a parallel hole collimator, a fan-beam collimator, a cone-beam collimator, a slit-slat collimator, a conical hole collimator, or the like, or any combination thereof.

For different QC items, the QC tools may be different. For example, for different QC items of the SPECT device, the radiation source or the phantom may be different. As a further example, if the QC item is the uniformity test, the QC tool may include the ^{99m}Tc flood source planar phantom; if the QC item is the spatial resolution test, the QC tool may include a lead grid phantom or a line source if the QC item is the sensitivity test, the QC tool may include a standard activity point source. As another example, if the QC item is the COR calibration, the QC tool may include a point source bracket, a ⁹⁹Tc point source and a laser locator. As still another example, if the QC item is the energy peak calibration, the QC tool may include a multi-energy radiation source bracket, a collimator adaptor, and energy spectrum analysis software.

In some embodiments, the relationship between QC tools and QC items may be stored in the storage device 130, and the processing device 120 may determine the one or more target QC tools based on the target QC item. The one or more target QC tools may include a target QC subject, a target QC fixture, and/or a target collimator.

In some embodiments, the one or more target QC tools may be determined based on an input of an operator via the interface of a QC application implemented on the terminal device. For example, the operator may select the one or more target QC tools via the interface of a QC application.

In 1330, the processing device 120 may cause the cart to transport at least one of the one or more target QC tools from a tool storage device to a target location.

In some embodiments, the tool storage device 160 may include a plurality of QC tools. Each QC tool in the tool storage device 160 may be labelled with an identifier. The identifier may include a mechanical switch, a proximity switch, a photoelectric switch, an electromagnetic sensor, a radiofrequency identification tag, a unique symbol (e.g., a QR code, a bar code, etc.), or the like, or any combination thereof. An identifier may be configured to distinguish different QC tools. The identifier may include type information, configuration information, model information, name information, ID number information, etc.

In some embodiments, the processing device 120 may identify, from the plurality of QC tools stored in the tool storage device 160, the target QC tools based on the identifiers labelled on the plurality of QC tools. For example, the information of the identifiers of the plurality of QC tools may be stored in the storage device 130. The processing device 120 may obtain a target identifier of a target QC tool from the storage device 130. The processing device 120 may identify a plurality of identifiers corresponding to the plurality of QC tools stored in the tool storage device 160 via the cart. The processing device 120 may then identify the target QC tool by matching the target identifier with one of the plurality of identifiers corresponding to the plurality of QC tools. The identification of the target QC tool may be the same as or similar to the identification of the target collimator, which is found elsewhere in the present disclosure. See, e.g., FIG. 10 and the descriptions thereof.

In some embodiments, the processing device 120 may send an identification request to the cart. The identification request may include the target identifier (e.g., the name, the ID number, the model or configuration information, etc.), of the target QC tool. The cart may identify the target QC tool from the tool storage device based on the target identifier.

Taking RFID tags as an example. Each QC tool in the tool storage device is affixed with an RFID tag. Each RFID tag may contain specific equipment information of the QC tool. For example, the RFID tag affixed to a collimator may include the model or specification data of the collimator. As another example, the RFID tag affixed to a QC fixture may include the type or configuration information of the QC fixture. As still another example, the RFID tag affixed to a phantom may include the name information of the phantom. As still another example, the RFID tag affixed to a radiation source may include the name information of the radiation source.

The cart may be equipped with an RFID reader. The RFID may include an RFID antenna and an antenna receiver. When the cart receives an identification instruction from the processing device 120, the cart may move within a certain range of the tool storage device. The RFID tags on the QC tools in the tool storage device may transmit their information (model/type data) to the antenna receiver via the RFID antenna. The antenna receiver may collect all tag information of the RFID tags on the QC tools and forward the tag information of the RFID tags on the QC tools to the processing device 120. The processing device 120 may compare the tag information of the RFID tags on the QC tools with the information of the target identifier of the target QC tool and determine one of the RFID tags on the QC tools that matches the information of the target identifier of the target QC tool. The processing device 120 may transmit the determined tag information of an RFID tag to the cart, and the cart may be controlled to grip the target QC tool from the tool storage device 160 based on the determined tag information of an RFID tag that matches the target identifier of the target QC tool.

In some embodiments, the cart may include an image sensor and a control unit. After the control unit receives the identification instruction from the processing device 120, the control unit may control the cart to acquire images of the plurality of QC tools in the tool storage device via the image sensor. The control unit may determine the target QC tool from the plurality of QC tools in the tool storage device based on the images of the plurality of QC tools. For example, the control unit may receive a reference image of the target QC tool and match the reference image with one of the images of the plurality of QC tools to identify the target QC tool from the plurality of QC tools in the tool storage device. The control unit may match the reference image with one of the images of the plurality of QC tools based on a similarity between the reference image and each of the images of the plurality of QC tools. In some embodiments, the control unit may perform an object identification on the images of the plurality of QC tools to identify the target QC tool. The control unit may be a portion of the processing device or separated from the processing device.

In some embodiments, each of the plurality of QC tools in the tool storage device may be located at a reference location, and the reference locations of the plurality of QC tools in the tool storage device may be stored in a storage unit of the cart. The control unit may determine the reference location of the target QC tool and identify the target QC tool from the plurality of QC tools based on the reference location. The storage unit may be a portion of the storage device 130 or separated form the storage device 130.

In some embodiments, the processing device 120 may use the cart 140 to grip the target QC tool from the tool storage device 160 and support the target QC tool. For example, the mechanical arm (e.g., the gripper) of the cart 140 may grip the target QC tool from the tool storage device 160 after identifying the target QC tool. In some embodiments, the processing device 120 may transport, using the retractable arm of the cart 140, the target QC tool to the cart body. For example, the processing device 120 may control the gripper to grip the target QC tool, and control the retractable arm to extend or retract to transport the target QC tool to the cart body. In some embodiments, the cart body (e.g., the collimator platform) may support the target QC tool during a transportation process of the target QC tool from the tool storage device 160 to one or more target locations.

In some embodiments, to facilitate the automatic installation of the target QC tool, the processing device 120 may use the cart 140 (e.g., the wheels of the cart 140) to transport a target QC tool from the tool storage device 160 to a target location of the medical device. The target location may be the same as or different from a target installation location of the target QC tool. The target installation location may include the surface of the detector, the surface of the collimator, a location near the surface of the detector or the collimator, a location on the rotation axis of the gantry of the medical device, or the bed. As used herein, A “near” B refers to a distance between A and B that is less than a threshold, e.g., 1 cm, 2 cm, 5 cm, 10 cm, etc.

The target location of a target QC tool may be a default setting of the system or set by an operator.

For example, the target location may be close to a detector of the medical scanner 111. As another example, the target location may be close to a bed (e.g., the bed 112) of the medical device. As still another example, the target location may be close to the radiation source of the medical device.

In some embodiments, the target location of the target QC tool and/or the predetermined route of the cart from the tool storage device and the target location may be a default setting of the system or may be set by the operator. The processing device 120 may cause the cart to move along the predetermined path after the cart grips the target QC tool.

In some embodiments, at least one of the one or more target QC tools may be transported by an operator from the tool storage device, such as the radiation source or the phantom.

In 1340, the processing device 120 (e.g., the control module 430) may automatically install, using the cart, the at least one of the one or more target QC tools.

As used herein, the word “automatically” may refer to the installation of the target QC tool being performed in a mechanical manner without any human assistance.

The processing device 120 may install, using the cart, at least one of the one or more target QC tools at a target installation location. The target install location of a target QC tool may be a default setting of the system or set by an operator.

The target installation location of a target QC subject (e.g., a radiation source or a phantom) may include the surface of the detector, the surface of the collimator, a location near the surface of the detector or the collimator, a location on the rotation axis of the gantry of the medical device, the bed of the medical device, etc. As used herein, A “near” B refers to a distance between A and B that is less than a threshold, e.g., 1 cm, 2 cm, 5 cm, 10 cm, etc. The threshold may be a default setting of the system or set by an operation of the system.

In some embodiments, for different QC items, the target installation location of the target QC subject (e.g., the radiation source or phantom) may be different. For example, for the uniformity test, a phantom may be arranged on the surface of the detector using Nylon clamps or a magnetic fixture; for the intrinsic resolution test, a point source or a line source may be arranged at 1-5 cm from the surface of the detector of the medical device via a bracket; for the system resolution test, a line source or a lead grid phantom may be arranged at 10 cm from the surface of a collimator of the medical device via a bracket; for the COR calibration, a point source may be arranged at a target install location on a rotation axis of the gantry of the medical device; for the sensitivity test, a point source may be arranged at 10 cm from the surface of the detector of the medical device via a bracket.

In some embodiments, the target QC subject (e.g., the radiation source or the phantom) used for the target QC item may be positioned on the bed (e.g., bed 112) of the medical device, or directly positioned on the cart. The bed or the cart provided with the target QC subject (e.g., the radiation source or the phantom) may be controlled to move to the imaging field of the medical device. For example, the target QC subject (e.g., the radiation source or the phantom) used for the target QC item may be positioned on the bed or the cart by an operator. As another example, the target QC subject (e.g., the radiation source or the phantom) used for the target QC item may be positioned on the bed or the cart by the cart automatically.

In some embodiments, multiple radiation sources may be located on the bed. Generally, the QC process needs to be carried out multiple times, or a QC process may include multiple QC items that need multiple radiation sources or phantoms, and the radiation sources or phantoms used each time or QC item may be different. Therefore, to improve the efficiency of quality control, all the required radiation sources can be placed on the bed at one time. The placement positions of different radiation sources are different. According to the pre-marked positions of different radiation sources, each radiation source can be placed at a different position on the bed.

In some embodiments, if the target QC tool includes a target collimator, the target collimator may be arranged at the surface of the detector, a location near an X-ray tube of the CT device, a location within the treatment head of a treatment device, etc.

In some embodiments, if the target QC tool includes a target QC fixture, the target QC fixture may be located near the target QC subject.

In some embodiments, the processing device 120 may automatically install, by a locating module of the cart 140, the target QC tool (e.g., the target QC subject) to the target installation location (e.g., the surface of the detector or the collimator of the medical device). In some embodiments, the locating module may include a locating hole, a locating pin, a bolt, a nut, or the like, or any combination thereof. In some embodiments, the locating accuracy of the locating module may be 0.25 millimeter level. For example, the locating module of the cart 140 may include a locating hole, and the medical device may include a locating pin that is paired with the locating hole. As another example, the locating module of the cart may include a locating pin, and the medical device may include a locating hole that is paired with the locating pin. The locating hole may match with the locating pin, and the processing device 120 may control the gripper of the cart 140 to mount the target QC tool onto the medical device. In some embodiments, the target QC tool may be installed by fixedly connecting with the detector or the collimator of the medical device.

In some embodiments, the target QC tools may include a target collimator, the processing device 120 may use the cart 140 to transport, based on a predetermined route, the target collimator from the tool storage device 160 to the predetermined location of the medical device. In some embodiments, the processing device 120 may use the cart 140 to align, based on a navigation algorithm, the target collimator with the detector of the medical device. For example, the cart 140 may align the target collimator with a fixture of the detector to facilitate the automatic installation of the target collimator. In some embodiments, the alignment accuracy of the alignment based on the navigation algorithm may be at the millimetre level. More descriptions regarding transporting and aligning the target QC tool may be the same as or similar to the target collimator and may be found elsewhere in the present disclosure. See, e.g., FIG. 9 and the descriptions thereof.

In some embodiments, after the target collimator is arranged at the target installation location, other target QC tools may be installed at the target installation location. For example, the target QC subject (e.g., a phantom and/or a radiation source) may be installed at the target installation location.

In some embodiments, the target QC subject may be installed at the target installation location before the installation of the target collimator.

In some embodiments, if the target QC item requires no collimator, before installing the target QC tools, the medical device may include a pre-installed collimator that is already installed. The processing device 120 may automatically uninstall the installed collimator before installing the target QC tools. For example, the processing device 120 may automatically uninstall, using the cart 140, the installed collimator from the medical device based on the installation instruction. For example, after the cart 140 transports a target QC tool to the predetermined location of the medical device and before installing the target QC tool, the processing device 120 may control the cart 140 to uninstall the installed collimator. For example, the gripper of the cart 140 may grip the installed collimator and transport the installed collimator from the medical device to the cart 140 (e.g., the installed collimator may be transported to the collimator platform of the cart 140). In some embodiments, the processing device 120 may use the cart 140 to transport the installed collimator from the medical device to the tool storage device 160. For example, after uninstalling the installed collimator and installing the target collimator, the cart 140 may transport the installed collimator to the tool storage device 160. In some embodiments, the installation of the target QC tool and the uninstallation of the installed collimator may be performed by the same cart 140. Alternatively, the installation of the target QC tool and the uninstallation of the installed collimator may be performed by two different carts 140. For example, one cart may be configured to install the target QC tools, and the other cart may be configured to uninstall the installed collimator. More descriptions regarding uninstalling the installed collimator may be found elsewhere in the present disclosure. See, e.g., FIG. 11 and the descriptions thereof.

In some embodiments, before installing the required target QC tool to the target installation location (e.g., the surface of the detector or the collimator, or a location near the surface of the detector or the collimator), the cart may determine whether other components are mounted to the target installation location. If other components are mounted to the target installation location, the cart may remove and return them to the tool storage device before installing the currently target QC tool.

Based on this, in an exemplary embodiment, installing a target QC tool may include sending a historical component detection request to the cart by the processing device, instructing the cart to check whether one or more historical components are mounted on the target installation location. If the one or more historical components are detected at the target installation location, the cart may be controlled to remove the one or more historical components from the target installation location and install the target QC tool to the target installation location. The one or more historical components may include a historical collimator, a historical QC fixture, a historical radiation source or phantom, etc.

After transporting the target QC tool to the target location, the processing device may send the historical component detection request to the cart to check whether a historical component (e.g., a QC tool) is mounted on the target installation location. For example, the cart may use RFID tags to detect whether a historical component is mounted on the target installation location. Upon reaching the target installation location, the cart may continue to receive information via the RFID antenna and the antenna receiver. If the antenna receiver does not detect any collimator model or fixture type information other than that of the target QC tool (e.g., a target collimator or target QC fixture) within a preset time, it indicates no historical components are mounted. If other collimator model or fixture type information is received, it confirms the presence of a historical component (e.g., a historical collimator or historical quality control fixture). In this case, the cart may use its robotic arm to remove the historical component and install the target QC tool (e.g., a target collimator or target quality control fixture). After installation, the historical component is loaded onto the cart, transported back to the tool storage device, and returned to its designated position.

In the embodiments of the present disclosure, the QC process is categorized into multiple types based on the installation status of required components, including: QC with no collimator and no QC fixture, QC with no collimator but with a QC fixture, QC with a collimator but no QC fixture. Further subdivisions can be made based on different collimator models and fixture types.

For these different QC processes, the required components must be handled according to the previous and current processes. For example, if the last process involved no collimator and no QC fixture, and the current process requires no collimator but QC Fixture A, the cart may receive a fixture identification request carrying type information of QC Fixture A's type information. The cart may move to the tool storage device, identify QC Fixture A, load QC Fixture A onto the designated position, and transport QC Fixture A to the target installation location for mounting. Before mounting QC Fixture A, the cart may determine whether any historical components are present. Since the previous process had no mounted components, QC Fixture A is directly installed.

After successful installation, the detector acquires scan images to determine the current QC result. Subsequent processes (e.g., quality control with Collimator A and no fixture) follow the same workflow: the cart identifies and transports new QC tools used for a next QC item, removes historical QC tools used for a previous QC item if present and transport the historical QC tools to the tool storage device, and installs the new QC tools to execute the next QC item until completion.

In the quality control method for a medical device provided by these embodiments, the cart checks for historical components before mounting the target QC tool. If detected, it removes them first, streamlining the workflow by avoiding redundant trips for component replacement. This optimization simplifies operations, enhances efficiency, and ultimately improves the overall quality control process.

In some embodiments, at least one of the one or more target QC tools may be installed by an operator from the tool storage device, such as the radiation source or the phantom. The operator may be arranged the radiation source or the phantom on the bed or the cart.

In 1350, the processing device 120 may perform the target QC item to obtain a QC result based on the installed target QC tool.

In some embodiments, the processing device 120 may control the medical device to scan the target QC subject (e.g., a radiation source and/or phantom) to obtain image data, and determine the QC result based on the image data.

In some embodiments, the target QC item may include a test item, and the QC result may include one or more QC indices determined based on image data. For each QC item, one or more QC indexes may be used to evaluate or verify the performance of the medical device associated with the target QC item. For example, for the SPECT device, in the uniformity test, an integral uniformity and an differential uniformity may be used to evaluate the uniformity of the medical device; in the spatial resolution test, a full width at half maximum (FWHM) and a modulation transfer function (MTF) corresponding to 10% of the spatial frequency may be used to evaluate the spatial resolution of the medical device; for the sensitivity test, a detector count rate per unit activity and a loss of dead time may be used to evaluate the sensitivity of the medical device; for the COR calibration, a COR offset may be used to calibrate the COR of the medical device.

A QC index corresponding to the target QC item may be determined based on the image data and/or the reconstructed image according to a predetermined algorithm, a trained machine learning model, etc. For example, the processing device may extract image features from the reconstructed image and determine one or more QC indices based on the extracted image features. The image features may include a texture, the shape of the radiation source or phantom, the energy spectrum response, etc.

In some embodiments, the QC result may include whether a QC index corresponding to the target QC item involves an anomaly. In some embodiments, if the QC index corresponding to the target QC item involves an anomaly, the QC result may also include an anomaly reason and/or a solution of the anomaly. In some embodiments, the processing device 120 may compare the QC index determined based on the reconstructed image with a reference value or range. If the QC index determined based on the reconstructed image is different from the reference value or not within the reference range, the processing device 120 may determine that the QC index is involved an anomaly.

In some embodiments, the processing device 120 may determine the anomaly reason and/or a solution of the anomaly based on the QC index and/or historical data of the medical device using a trained machine learning model. The historical data of the medical device may include the historical QC indices corresponding to the target QC item and/or the anomaly reasons and solutions corresponding to the historical anomalies in the history QC indices. The trained machine learning model may be a root cause analysis model (e.g., a 5-why analysis model, a fishbone diagram model, etc.).

In some embodiments, different QC indexes may correspond to different reference anomaly reasons and corresponding solutions. The processing device 120 may determine the anomaly reason and/or a solution of the anomaly based on the QC index and the reference anomaly reasons and corresponding solutions.

In some embodiments, the processing device 120 may determine whether the medical device involves an anomaly based on the reconstructed image, and the QC result may include a determination of whether the medical device involves an anomaly. For example, the processing device 120 may identify or segment the target QC subject (e.g., the radiation source or a phantom) from the reconstructed image and determine whether the medical device involves an anomaly by comparing the identified or segmented target QC subject with a reference image of the target QC subject. If a difference between the identified target QC subject and the reference image of the target QC subject exceeds a threshold, the processing device may determine that the medical device involves an anomaly.

For example, if the phantom includes a lead grid phantom including multiple parallel seams, the image data of the lead grid phantom may be obtained by the detector of the medical device, and an image of the lead grid phantom may be reconstructed based on the image data. The processing device 120 may determine whether the parallel lead seams in the image are normal. If the lead seams in the image of the lead grid phantom are curved, it indicates the medical device involves an anomaly that should be determined as a QC result for the medical device.

In some embodiments, the QC item may include a calibration item, e.g., the COR calibration, a multi-probe registration, a pixel size calibration, a sensitivity calibration, an attenuation calibration, a spatial resolution calibration, a spatial uniformity calibration, a spatial linearity calibration, a gantry and probe tilt calibration, an energy linearity calibration, an energy resolution, etc. The QC result may include a calibration parameter. For example, for the COR calibration, the calibration parameter may include a COR offset. As another example, for the sensitivity calibration, the calibration parameter may include a sensitivity map. As still another example, for the attenuation calibration, the calibration parameter may include a u-map. It should be noted that in some embodiments, the calibration item and the test item may belong to the same QC process. For example, after performing the test item, the processing device 120 may determine whether a calibration item needs to be performed (i.e., whether the medical device needs to be calibrated) based on the test result. If the test result indicates that the medical device is abnormal, the calibration item needs to be performed. The test item may be performed to determine whether the performance of the medical device meets a standard, and the calibration item may be performed to adjust one or more device parameters of the medical device to make the performance of the medical device meet the standard.

In some embodiments, a calibration parameter of the medical device may be determined based on the reconstructed image or the image data. When performing the calibration item, after obtaining the image data, the corresponding calibration parameters may be determined based on the reconstructed image or the image data according to the calibration item. For example, taking energy resolution as an example, energy information is extracted from the reconstructed image, and then the energy resolution parameter is determined based on the energy information. This energy resolution parameter serves as the calibration parameter for energy resolution. As another example, taking the COR calibration as an example, whether the COR is normal may be determined based on the image data (e.g., a sinogram presenting the image data). If the sinogram presenting the image data involves fracture or fluctuation, the COR is normal. The COR offset may be determined based on the image data. Then the installation bolts of the detector or gantry may be adjusted or the COR offset may be compensated based on software.

In some embodiments, the calibration item may be performed by adjusting one or more device parameters of the medical device (e.g., the COR) based on the calibration parameters to obtain the calibration result. In some embodiments, the QC result may include the calibration result. The calibration result may include the updated device parameters (e.g., a compensation value of the COR).

Using energy resolution as an example, after obtaining the energy resolution parameter, the energy window width and energy center of the medical device may be adjusted based on the difference between this energy resolution and the expected energy resolution. The adjusted energy window width and energy center may be then recorded as the energy resolution calibration results of the medical device.

In the QC process for a medical device provided by the embodiments of this application, quality control includes device calibration, and the QC result includes a calibration result. The calibration parameters of the medical device are determined based on the reconstructed images, and then the device's parameters are adjusted according to these calibration parameters to obtain the device calibration results. In this method, during device calibration, the corresponding calibration parameters are calculated by processing the scanned images. Based on these calibration parameters, the relevant device parameters of the medical scanning device are adjusted to restore the device to normal operation, thereby improving the stability of the medical scanning device.

According to some embodiments of the present disclosure, based on the image, the system performs a status assessment of the medical device by determining the QC indices based on the reconstructed image. This enables rapid acquisition of the status information of the medical device. This approach facilitates timely detection of abnormalities of the medical device and prompt intervention, thereby enhancing both the reliability of the medical device and the quality of imaging output.

In some embodiments, the quality control process needs to be carried out multiple times, or multiple QC items may be performed in order. Before performing a current QC item, the processing device 120 may automatically uninstall the installed QC tool in the previous QC item before installing the target QC tools in the current QC item. For example, the processing device 120 may automatically uninstall, using the cart 140, the installed QC tool from the medical device based on the installation instruction. For example, after the cart 140 transports the target QC tools used in the current QC item to the predetermined location of the medical device and before installing the target QC tools in the current QC item, the processing device 120 may control the cart 140 to uninstall the installed QC tool used in the previous QC item. For example, the gripper of the cart 140 may grip the installed QC tool used in the previous QC item and transport the installed QC tool used in the previous QC item from the medical device to the cart 140 (e.g., the installed collimator may be transported to the collimator platform of the cart 140). In some embodiments, the processing device 120 may use the cart 140 to transport the installed QC tool used in the previous QC item from the medical device to the tool storage device 160. For example, after uninstalling the installed QC tool used in the previous QC item and installing the target QC tool used in the current QC item, the cart 140 may transport the installed QC tool used in the previous QC item to the tool storage device 160. In some embodiments, the installation of the target QC tool in the current QC item and the uninstallation of the installed QC tool used in the previous QC item may be performed by a same cart 140. Alternative, the installation of the target QC tool and the uninstallation of the installed QC tool used in the previous QC item may be performed by two different carts 140. For example, one cart may be configured to install the target QC tools in the current QC item, and the other cart may be configured to uninstall the installed QC tool used in the previous QC item. More descriptions regarding uninstalling the installed collimator may be found elsewhere in the present disclosure. See, e.g., FIG. 11 and the descriptions thereof.

In the quality control method for a medical device provided by the embodiments of this application, in response to obtain a target QC item or a QC request for a medical device, based on the target QC item, the cart is controlled to identify the target QC tools required for quality control and install these target QC tools on the medical device. The detector is controlled to acquire image data of the radiation source and/or phantom, and the QC result of the medical device is determined based on the image data by the processing device.

In some embodiments, if the radiation source and/or the phantom is arranged on the bed of the medical device, the bed of the medical device may be controlled to move until the radiation source and/or the phantom on the bed is positioned within the imaging field of view of the detector in the medical device.

In this method, during the quality control process of the medical device, the bed is automatically adjusted to a position where the radiation source within the detector's imaging field of view, allowing its emitted radiation to reach the detector for image acquisition. Simultaneously, the cart identifies the required QC tools and installs or removes them to the target installation location. By analyzing the reconstructed image obtained from the radiation source and/or phantom, the QC result of the medical device is determined.

The method for QC provided by some embodiments of the present disclosure simplifies the placement, installation, and removal of necessary QC tools by directly controlling the cart or the bed to identify, transport, and install the QC tools. As a result, the detector can quickly and accurately acquire scan images, enabling rapid determination of the medical device's quality control results and improving operational efficiency. Moreover, the entire QC process is fully automated, eliminating the need for manual intervention, reducing time costs, and further enhancing operational efficiency.

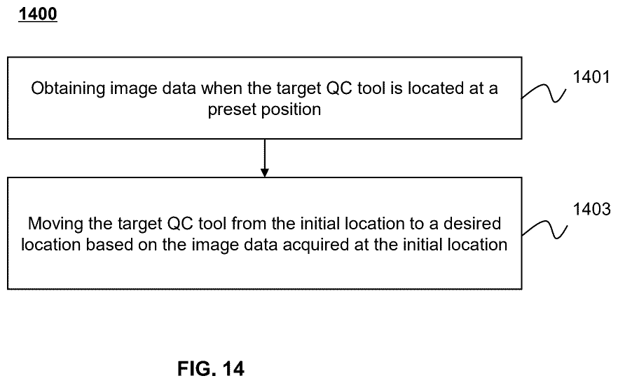

FIG. 14 is a flowchart illustrating an exemplary process for automatically adjusting a position of a QC tool according to some embodiments of the present disclosure. In some embodiments, the process 1400 may be implemented in the automatic installation system 100 illustrated in FIG. 1. For example, the process 1400 may be stored in the storage device 130 and/or the storage (e.g., the storage 220, the storage 390) as a form of instructions, and invoked and/or executed by the processing device 120 (e.g., the processor 210 of the computing device 200 as illustrated in FIG. 2, the CPU 340 of the mobile device 300 as illustrated in FIG. 3). The operations of the illustrated process presented below are intended to be illustrative. In some embodiments, the process 1400 may be accomplished with one or more additional operations not described, and/or without one or more of the operations discussed. Additionally, the order in which the operations of the process 1400 as illustrated in FIG. 13 and described below is not intended to be limited.

In some embodiments, after the cart transports a target QC tool from the tool storage device and install the target QC tool at a target installation location, the target installation location of the target QC tool may have an error with a desired location, then the target installation location of the target QC tool may be adjusted for acquiring an image with improved quality. For example, the target QC tool includes a radiation source or a phantom. The radiation source or the phantom may be transferred from the tool storage device to the bed of the medical device at an initial location. The location of the radiation source or the phantom (i.e., the location of the bed) may be further adjusted according to process 1400 in FIG. 14. As another example, the radiation source or the phantom may be placed at a target installation location near the surface of the detector or collimator. If the image of the radiation source or the phantom at the target installation location does not satisfy a condition, then the target installation location may be adjusted.

In 1401, when a target QC tool (e.g., a radiation source or phantom) is located at a preset position, image data may be obtained.

In some embodiments, the preset position may be the same as the target location where the cart transports the target QC tool for installation.

In some embodiments, the target QC tool may be the radiation source or the phantom and arranged on the bed of the medical device or directly placed on the cart. The preset position of the target QC tool refers to the initial location of the bed or the cart when the radiation source or the phantom is placed on the cart. After moving the bed to this initial location, further adjustments are required to precisely position the bed at the designated location.

In some embodiments, the preset position may be the same as the target installation location where the target QC tool is installed.

In some embodiments, whether the target QC tool has reached the preset position may be determined. If the target QC tool is the radiation source or the phantom and arranged on the bed of the medical device or directly placed on the cart, whether the target QC tool has reached the preset position may include whether the bed or cart has reached the preset position.

In some embodiments, a laser locator may be used to determine whether the target QC tool has reached the preset position. For example, the laser locator may be installed on the bed. As the bed moves, the laser beam emitted by the laser locator is projected onto a reference object (e.g., the detector), and the current position of the bed is determined based on the reflected signal. The current position may be then compared with the preset position. If the current position is matched with the preset position, the processing device may determine that the bed has reached the preset position. The current position matching the preset position refers to that a distance between the current position and the preset position is less than a threshold (e.g., 1 cm, 0.5 cm, etc.).

After the target QC tool (e.g., a radiation source or phantom) is at the preset position, the detector may be controlled to acquire image data of the target QC tool, and a candidate scan image may be obtained based on the image data. This image may be used to fine-tune the position of the target QC tool. It should be noted that some medical devices may have no detector, such as an MRI device, in this case, an RF receiving coil may be controlled to acquire image data (e.g., MR signals).

For example, after the bed moves to the preset position, the radiation source or phantom may move to the preset position, the detector may be controlled to acquire image data of the radiation source or phantom, and a candidate scan image of the radiation source or phantom may be obtained based on the image data.

In 1403, the target QC tool may be moved from the initial location to a desired location based on the image data acquired at the initial location. When the target QC tool is positioned at the desired location, a condition may be met.

In some embodiments, the desired location may be a location different from the target installation location of the target QC tool.

In some embodiments, the desired location may be a location the same as the target installation location of the target QC tool.

In some embodiments, the condition being satisfied may include that the radiation source or the phantom is within the imaging field of view of the detector.

In the embodiments of this application, the imaging field of view of the detector refers to the precise target location of the radiation source, where the acquired image achieves the highest quality. Therefore, after moving the scanning bed to the initial location, further adjustments are necessary to ensure the radiation source is accurately positioned.

Taking the radiation source or the phantom being placed on the bed or cart as an example, an initial image of the radiation source or the phantom may be generated based on the image data of the radiation source or the phantom when the bed or cart is positioned at the initial location. The position of the bed or the cart may be adjusted based on the location information of the radiation source or the phantom in the initial image.

For example, the spatial position information of the radiation source or the phantom may be determined from the initial image. The spatial position information may be compared with the desired position of the radiation source or the phantom. The position of the bed or the cart may be adjusted based on the deviation between the spatial position information and the desired position of the radiation source or the phantom, ensuring the radiation source or the phantom is correctly positioned within the detector's imaging field of view.

The spatial position information refers to the actual physical location of the radiation source or the phantom.

In some embodiments, the processing device 120 may control the target QC tool to move a distance to a candidate location, and then candidate image data may be acquired by the medical device when the target QC tool is at the candidate location. The distance may be a default setting of the system or set by an operator. Then the processing device 120 may determine whether the quality of the image satisfies a condition. In response to determining that the quality of the image satisfies the condition, the candidate location may be the desired location of the target QC tool, and then the target QC item may be performed. In response to determining that the quality of the candidate image data does not satisfy the condition, the processing device 120 may control the target QC tool to move a distance from the candidate location to a next candidate location. Then next candidate image data may be acquired by the medical device when the target QC tool is at the next candidate location. Then, the processing device 120 may determine whether the quality of the next candidate image data meets a specified condition. The location of the target QC tool may be adjusted as described above until it is positioned at the desired location, where the quality of the image data acquired meets the specified condition. The condition may be associated with a quality index, such as the noise level, the resolution, etc.

The initial image may include the position of the radiation source or the phantom in the initial image. Using a predefined transform relationship between an image coordinate system and a world spatial coordinate system, the spatial position information in the initial image may be transformed into real-world spatial coordinates in the world spatial coordinate system. By comparing these real-world spatial coordinates with the desired position of the radiation source or the phantom, the bed or the cart may be moved accordingly to align the radiation source or the phantom precisely with the desired location.

According to some embodiments of the present disclosure, using a predefined transform relationship between an image coordinate system and a world spatial coordinate system, position information in the initial image may be transformed into position information in space (i.e., real-world spatial position or actual spatial position). Based on the deviation between this real-world spatial position and the desired position of the radiation source or the phantom, the bed or the cart may be further adjusted to align the actual spatial position of the radiation source or the phantom with the desired position. This ensures precise placement of the radiation source or the phantom at the desired location, thereby improving both the efficiency and accuracy of its positioning. As a result, more accurate and higher-quality images can be acquired, ultimately enhancing the reliability of the QC result.

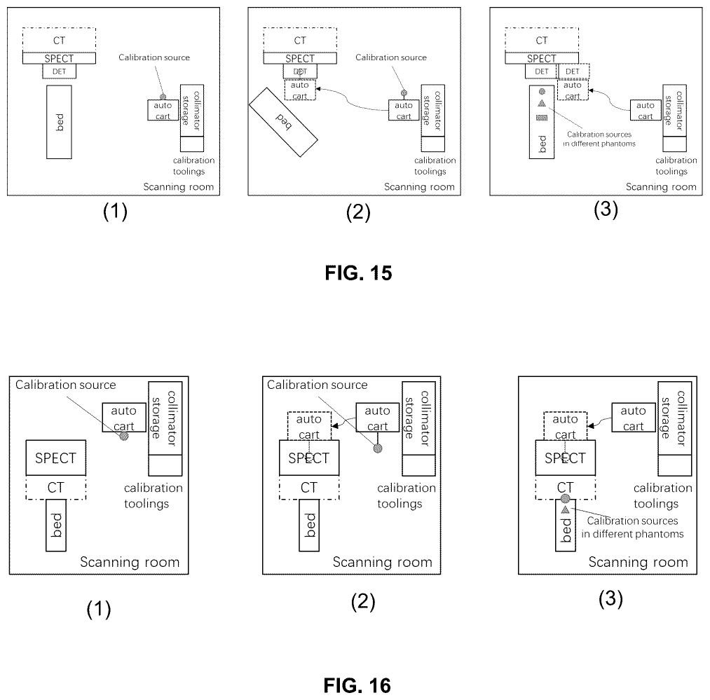

FIG. 15 is a diagram illustrating an exemplary process for automatically QC according to some embodiments of the present disclosure.

As shown in FIG. 15, taking QC with a collimator as an example. In (1), it can be seen that neither the collimator nor the QC fixture is mounted on the detector. The cart identifies the required collimator from the tool storage device. Then in (2), the cart moves to the detector location with the collimator and installs the collimator onto the detector, and the bed moves away from the detector location for the cart. In (3), after the collimator installation, the bed is controlled to move for positioning the radiation source within the imaging field of view of the detector in the medical device.

In this embodiment, by controlling the cart to identify and operate the required quality control tool, and directly controlling the bed to position the radiation source at the designated location, the operational procedures for placing, installing, and removing necessary components are simplified. This enables the detector to quickly and accurately acquire scan images, thereby improving the operational efficiency of quality control. Moreover, the entire quality control process is fully automated, eliminating the need for manual intervention, which reduces time costs and further enhances the operational efficiency of quality control.

FIG. 16 is a diagram illustrating an exemplary process for automatically QC according to some embodiments of the present disclosure

As shown in FIG. 16, taking QC without a collimator as an example. In (1), it can be seen that neither the collimator nor the QC fixture is mounted on the detector. The cart identifies the required radiation source or phantom from the tool storage device. Then in (2), the cart moves to the detector location with the required radiation source or phantom and installs the required radiation source or phantom onto the bed and the bed does not need to move away from the detector location for the cart. In (3), after the required radiation source or phantom is placed on the bed, the cart is controlled to transport another radiation source or phantom to the bed.

In this embodiment, by controlling the cart to identify and operate the required quality control tool, and directly controlling the bed to position the radiation source at the designated location, the operational procedures for placing, installing, and removing necessary components are simplified. This enables the detector to quickly and accurately acquire scan images, thereby improving the operational efficiency of quality control. Moreover, the entire quality control process is fully automated without requiring manual intervention, which reduces time costs and further enhances the operational efficiency of quality control.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. An automatic installation system, comprising a cart, a storage medium including a set of instructions, and at least one processor configured to communicate with the storage medium, wherein when executing the set of instructions, the at least one processor is configured to direct the system to perform operations including:
determining a target quality control (QC) item for a medical device;
determining one or more target QC tools based on the target QC item;
using a cart to transport at least one of the one or more target QC tools from a tool storage device to a target location;
automatically installing, using the cart, the at least one of the one or more target QC tools; and
performing the target QC item to obtain a QC result based on the installed target QC tool.

2. The automatic installation system of claim 1, wherein using a cart to transport at least one of the one or more target QC tools from a tool storage device to a target location includes:
identifying, from a plurality of QC tools in the tool storage device using the cart, the at least one of the one or more target QC tools.

3. The automatic installation system of claim 2, wherein the identifying, from a plurality of QC tools in the tool storage device using the cart, the at least one of the one or more target QC tools includes:
obtaining a target identifier of the at least one of the one or more target QC tools;
identifying a plurality of identifiers respectively corresponding to the plurality of QC tools; and
identifying the at least one of the one or more target QC tools by matching the target identifier with one of the plurality of identifiers respectively corresponding to the plurality of QC tools.

4. The automatic installation system of claim 3, wherein the plurality of identifiers of the plurality of QC tools is identified according to at least one of: a mechanical switch, a proximity switch, a photoelectric switch, an electromagnetic sensor, a radiofrequency identification tag, or a unique symbol.

5. The automatic installation system of claim 2, wherein the identifying, from a plurality of QC tools in the tool storage device using the cart, the at least one of the one or more target QC tools includes:
sending an identification instruction to the cart, the identification instruction including a target identifier of the at least one of the one or more target QC tools; and
in response to receiving the identification instruction, identifying, by the cart from a plurality of QC tools in the tool storage device, the at least one of the one or more target QC tools based on the target identifier.

6. The automatic installation system of claim 1, wherein the using a cart to transport at least one of the one or more target QC tools from a tool storage device to a target location includes:
using the cart to transport, based on a predetermined route, the at least one of the one or more target QC tools from the tool storage device to a predetermined location of the medical scanner; and
using the cart to align, based on a navigation algorithm, the at least one of the one or more target QC tools with the target location.

7. The automatic installation system of claim 6, wherein the navigation algorithm includes at least one of: a laser navigation algorithm, a visual navigation algorithm, or an inertial navigation algorithm.

8. The automatic installation system of claim 1, wherein the automatically installing, using the cart, the at least one of the one or more target QC tools includes:
automatically installing, by a locating module of the cart, the at least one of the one or more target QC tools on a target installation location.

9. The automatic installation system of claim 8, wherein the locating module includes a locating hole or a locating pin.

10. The automatic installation system of claim 1, wherein the automatically installing, using the cart, the at least one of the one or more target QC tools includes:
sending a historical component detection request to the cart;
in response to receiving the historical component detection request, checking, by the cart, whether one or more historical components are mounted on a target installation location;
in response to detecting the one or more historical components at the target installation location, uninstalling the one or more historical components from the target installation location by the cart and installing the at least one of the one or more target QC tools to the target installation location by the cart.

11. The automatic installation system of claim 1, wherein the operations further include:
automatically uninstalling, using the cart, an installed QC tool from the medical device; and
using the cart to transport the installed QC tool from the medical device to the tool storage device.

12. The automatic installation system of claim 1, wherein the performing the target QC item to obtain a QC result based on the installed target QC tool includes:
obtaining image data based on the installed target QC tool; and
determining the QC result based on the image data, the QC result including at least one of one or more QC indices, one or more calibration parameters associated with the target QC item, and a determination of whether a QC index corresponding to the target QC item involves an anomaly.

13. The automatic installation system of claim 1, wherein the one or more target QC tools includes at least one of: a target collimator, a target radiation source, a target phantom, or a target QC fixture.

14. The automatic installation system of claim 1, further comprising the tool storage device configured to store a plurality of QC tools, wherein the tool storage device is away from the medical device.

15. The automatic installation system of claim 1, wherein the at least one processor is configured to direct the system to perform operations including:
obtaining a QC request for performing the target QC item; and
determining the target QC item based on the QC request.

16. The automatic installation system of claim 1, wherein the target QC item includes a calibration item, and wherein the operations further include:
in response to determining that a test result obtained after performing a test item indicates that the medical device involves an anomaly, performing the calibration item.

17. The automatic installation system of claim 1, wherein the target QC item includes a test item, and wherein the operations further include:
in response to determining that the calibration item is performed, performing the test item.

18. The automatic installation system of claim 1, wherein the at least one processor is configured to direct the system to perform operations including:
obtaining image data when one of the one or more target QC tools is located at a preset position; and
moving the one of the one or more target QC tools from the initial location to a desired location based on the image data acquired at the initial location.

19. A cart, comprising a mechanical arm, a cart body, a processor, and a storage medium including a set of instructions, the processor configured to communicate with the storage medium, wherein when executing the set of instructions, the processor is configured to direct the cart to perform operations including:
determining a target quality control (QC) item for a medical device;
determining one or more target QC tools based on the target QC item;
using a cart to transport at least one of the one or more target QC tools from a tool storage device to a target location;
automatically installing, using the cart, the at least one of the one or more target QC tools; and
performing the target QC item to obtain a QC result based on the installed target QC tool.

20. An automatic QC method, comprising:
determining a target quality control (QC) item for a medical device;
determining one or more target QC tools based on the target QC item;
using a cart to transport at least one of the one or more target QC tools from a tool storage device to a target location;
automatically installing, using the cart, the at least one of the one or more target QC tools; and
performing the target QC item to obtain a QC result based on the installed target QC tool.
